(19) United States
(12) Patent Application Publication
(10) Pub. No.: US 2023/0313312 A1
(43) Pub. Date: Oct. 5, 2023

(54) METHODS FOR EARLY DETECTION OF BREAST CANCER

(71) Applicant: THE TRUSTEES OF INDIANA UNIVERSITY, Bloomington, IN (US)

(72) Inventor: Harikrishna NAKSHATRI, Zionsville, IN (US)

(21) Appl. No.: 18/024,488

(22) PCT Filed: Aug. 25, 2021
(86) PCT No.: PCT/US2021/047506

§ 371 Date: Mar. 2, 2023

Related U.S. Application Data
(60) Provisional application No. 63/074,101, filed on Sep. 3, 2020.

(51) Int. Cl. C12Q 1/6886; C12N 5/09

(52) U.S. Cl. CPC C12Q 1/6886; C12N 5/0693; C12Q 2600/158; C12Q 2600/156

(57) ABSTRACT

The present disclosure relates to methods of detecting cancer cells from a sample containing breast milk-derived cells.

### CROSS REFERENCE TO RELATED APPLICATIONS

This application claims priority to U.S. Provisional Application Ser. No. 63/074,101, filed Sep. 3, 2021, the contents of which are hereby incorporated in their entirety.

### TECHNICAL FIELD

The present disclosure relates to methods of detecting cancer cells, and specifically detecting breast cancer cells from breast milk-derived cells.

### BACKGROUND

Detection and treatment prior to lymphatic spread is associated with favorable outcomes in breast cancer. For example, in the case of women who have estrogen receptor alpha-positive (ERα+) breast cancer and are treated with anti-estrogens, recurrences occur at a steady rate from years 5 to 20 after diagnosis. However, this recurrence is dependent on the status of lymph nodes at the time of diagnosis. Distal recurrence is 13% in women without lymph node involvement but is 34% in women with four or more lymph nodes involved. Radiologic techniques remain the mainstay of breast cancer detection but these methods detect tumors above 5 mm in diameter. Unfortunately, these methods are still not always reliable, with high rate of false positives and false negatives. An examination of the Dutch national registry for the frequency of missed breast cancers in women participating in a high-risk MRI screening program has revealed the limitations of using only radiological techniques for diagnosis. Among 131 breast cancer cases with a negative MRI 0-24 months before cancer detection, 31% of cases had MRI detectable cancers that were missed, whereas 34% of cases showed minimal signs. Therefore, complementary molecular assays are needed to improve earlier cancer detection.

Breast milk can be one source of cancer cells as it contains a variety of cell types. Cells from breast milk have been cultured and characterized for stemness activity and have been suggested to be a source of cells for regenerative medicine. Earlier culturing methods favored the propagation of stem/basal and mesenchymal stem cells from the breast milk.

Since the majority of breast cancers likely originate from luminal progenitor cells, earlier methods are not ideal for the expansion of luminal and cancer cells from breast milk for robust characterization. We recently reported a breast epithelial culturing method that did not require feeder layer cells and allowed for the propagation of breast epithelial cells that phenotypically correspond to stem, luminal progenitor and mature/differentiated luminal cells.

### SUMMARY

We initiated this study upon the contribution of breast milk from a patient who was diagnosed with ER+/Progesterone Receptor (PR)-positive (ER+/PR+) cancer in her right breast while breast feeding. We determined whether the above technique can be used to propagate and characterize cancer cells in breast milk and show that breast milk contains cells that display cancer stem cell (CSC) properties and carry mutations that are enriched in breast cancer. Since incidence of breast cancer during pregnancy is 1 in 3000, is expected to increase due to delay in childbearing, and breast cancers diagnosed during pregnancy or postpartum tend to be highly metastatic, the method described here is useful for rapid characterization of cancer cells in these patients including identification of driver mutations and screening for drugs that may potentially target these mutations.

Radiologic techniques remain the main method for early detection for breast cancer and are critical to achieve a favorable outcome from cancer. However, more sensitive detection methods to complement radiologic techniques are needed to enhance early detection and treatment strategies. Using our recently established culturing method that allows propagation of normal and cancerous breast epithelial cells of luminal origin, flow cytometry characterization, and genomic sequencing, we show that cancer cells can be detected in breast milk. Cells derived from milk from the breast with cancer were enriched for CD49f+/EpCAM−, CD44+/CD24− and CD271+ cancer stem-like cells (CSC). These CSC carried mutations within the cytoplasmic retention domain of HDAC6, stop/gain insertion in MORF4L1, and deletion mutations within SWI/SNF complex component SMARCC2. CSC were sensitive to HDAC6 inhibitors, BET bromodomain inhibitors, and EZH2 inhibitors, as mutations in SWI/SNF complex components are known to increase sensitivity to these drugs. Among cells derived from breast milk of additional 10 women not known to have breast cancer, two of them contained cells that were enriched for the CSC phenotype and carried mutations in NF1 or KMT2D, which are frequently mutated in breast cancer. Breast milk-derived cells with NF1 mutations also carried copy number variations in CDKN2C, PTEN, and REL genes.

In some embodiments, the methods described herein can enable rapid cancer cell characterization including driver mutation detection and therapeutic screening for pregnancy/post-partum breast cancers. Furthermore, the methods can be used as a surveillance or early detection tool for women at high risk for developing breast cancer.

In some embodiments, the methods presented herein can detect cancer cells non-invasively, which increases the feasibility of using this method in a surveillance setting. Our previous studies have shown that culturing of primary cells does not introduce mutations; therefore, any mutations detected in milk-derived cells are not likely due to culturing artifacts. The methods have an inbuilt control (milk from the other breast without aberrations from the same woman), which should help to eliminate any culture-induced artifacts corrupting cancer-specific mutation detection as well as bias attributable to inter-individual variations in the genotype and phenotype of breast epithelial cells.

In some embodiments, the methods described herein are useful for screening women who are considered to be at a higher risk of developing breast cancer due to family history, germ line breast cancer risk alleles, and dense breasts that hinder radiologic detection. Additionally, since breast milk is enriched for cells with proliferative capacity, the methods can help in cataloguing somatic mutation rates in BRCA1/BRCA2 mutation carriers or any other risk alleles associated with impaired DNA repair pathways.

### DESCRIPTION OF THE DRAWINGS

FIGS. 1A-1D: Cells derived from milk of the breast with cancer show enrichment of cells with CSC phenotype. FIG. 1A shows phase contrast images of cells derived from the breast milk. FIG. 1B shows cell surface marker profiles of cells from breast milk without (left) and with (right) cancer. Numerical values of different cell populations from two biological replicates are shown at the bottom. FIG. 1C shows cells from the right breast milk generated larger size mammospheres. FIG. 1D shows mammospheres of cells from the right breast milk were enriched for luminal progenitors (CD49f+/EpCAM+) compared to mammospheres of cells from the left breast milk.

FIG. 2: Cells grown from tissues of the breast with cancer after lumpectomy and chemotherapy treatment are enriched for mesenchymal stem-like cells. Tissues from mastectomy after successful treatment were cultured under the same condition as milk and resulting cells were characterized for cell surface markers. Cells derived from the right breast had no EpCAM+ cells and likely enriched for mesenchymal stem cells (CD44+, CD90+/CD73+) and/or basal/myoepithelial cells (CD10+/EpCAM−), whereas cells from the left breast had significant levels of CD49f+/EpCAM+ luminal progenitor cells.

FIGS. 3A-3B: Presence of cells enriched for CSC-like properties in breast milk of two donors. FIG. 3A shows cell surface marker profiles of the left and the right breast milk-derived cells from the donor M2. Although this donor is clinically not known to have breast cancer, cells derived from the right breast milk were enriched for CD49f+/EpCAM+, CD271+/EpCAM+, and CD44+/CD24− cells compared to cells from the left breast milk. Mammospheres generated by cells from the right breast milk showed elevated luminal progenitor cells (CD49f+/EpCAM+) compared to cells from the left breast milk, although there were no differences in size of mammospheres. FIG. 3B shows left breast milk-derived cells of donor M10 display CSC-like properties. Cell surface marker profiling showed a trend towards enrichment of CD271+/EpCAM− subpopulation in the left breast milk-derived cells compared to the right breast milk-derived cells of this donor. Left breast milk-derived cells also generated larger mammospheres and these mammospheres were enriched for luminal progenitor cells (CD49f+/EpCAM+) cells compared to cells from the right breast milk.

FIG. 4: Breast milk-derived cells from the left and the right breasts of not all donors show phenotypic differences. Phenotype of breast epithelial cells derived from breast milk of donors M3 and M5. There were only marginal differences in phenotype and in both cases, the mammosphere size was <30 micrometer.

FIGS. 5A-5C: Milk-derived cells express luminal-differentiated cell enriched genes. FIG. 5A shows MDS plot of RNA-seq data of cells derived from breast milk of six donors. Note RNA expression differences between cells of the right and the left breast milk irrespective of whether one of the breasts had cancer (M1) or phenotypically aberrant cells (M2 and M10). FIG. 5B shows qRT-PCR analyses of RNA from milk-derived cells for the expression of FOXA1 and GATA3. RNAs from milk-derived cells were analyzed in biologic triplicates. FIG. 5C shows left breast milk-derived cells of M10 express high levels of stemness-associated protein TP63 compared to the right breast milk-derived cells. TP63 expression was also higher in the right breast milk-derived cells of M1. Cell extracts obtained from three independent batch of cells were analyzed in case of M1.

FIGS. 6A-6C: Milk-derived cells of M1-right, M2-right and M10-left breasts contain driver mutations. FIG. 6A shows summary of somatic mutation patterns revealed through whole genome sequencing of DNA from indicated samples. DNA from blood of individual donor was sequenced for comparison. Mutation frequency in DNA was similar in breast milk-derived cells with and without enrichment of cells with cancer stem cell phenotype. FIG. 6B shows schematic view of few of the driver mutations observed in cells from M1-right, M2-right and M10-left breast milk-derived cells. Various characterized domains of the corresponding proteins are indicated. FIG. 6C shows frequency of mutations in HDAC6, SMARCC2, NF1 and KMT2D in breast cancer. Data were generated using the cBioportal database.

FIGS. 7A-7B: Sensitivity of right breast milk-derived cells of donors M1 and M2 to targeted therapies. FIG. 7A shows CNV analysis of DNA from breast milk-derived cells of M1, M2 and M10. CNVs in DNA from breast milk-derived cells of right of M1, right of M2 and left of M10 were determined by comparing to DNAs from breast milk-derived cells of left of M1, left of M2 and right of M10. Horizontal line indicates normal copy number value of 2. Genes with numbers close to 2.6 or above are considered amplified (indicated in red in case of M2-Right). Raw values for all 87 genes are provided in Tables 1, 2, and 3. FIG. 7B shows cells were treated with indicated drugs for five days and cell proliferation was measured using bromodeoxyuridine incorporation-ELISA. Results of two independent experiments at different concentrations of GSK126 and JQ1 are shown. Data shown are average and standard errors of six technical replicates. *p<0.001. **<0.02, untreated versus drug treated cells.

FIGS. 8A-8D: Experimental design and cell surface marker profiles of breast milk-derived cells of donor M4. FIG. 8A is a schematic view of the experimental design. FIG. 8B shows representative flow cytometry patterns of breast milk-derived cells of M4. FIG. 8C shows bar graphs showing similar CD49f/EpCAM and CD271/EpCAM staining patterns of breast milk-derived cells of this donor. FIG. 8D shows representative isotype antibody control staining patterns.

FIG. 9: Phenotypic similarities in cells derived from left and right breast milk of two other donors (M6 and M7).

FIG. 10: Phenotypic similarities in cells derived from the left and the right breast milk of two other donors (M8 and M9). Note inter-individual differences in cell surface marker profiles, particularly for CD201/EpCAM and CD271/EpCAM.

FIGS. 11A-11B: Breast milk-derived cells generate mammospheres FIG. 11A shows CD49f/EpCAM staining patterns of mammosphere-derived cells from M3 and M5. FIG. 11B shows growth rate of breast milk-derived cells of M2 and M10. *p<0.003; **p<0.0001 compared to proliferation rate at day 2.

### DETAILED DESCRIPTION OF THE INVENTION

The present disclosure is generally related to methods of collecting, extracting, and analyzing DNA in a breast milk-derived cell. In particular, methods are provided in accordance with the present disclosure for collecting, extracting, and analyzing DNA in a breast milk sample comprising cancer stem-like cells. In some embodiments, a method is provided for culturing breast milk-derived cells.

In some embodiments, the breast milk is mixed with media and plated directly on a 804G conditioned media coated plate. In some embodiments, the milk to media ratio is 1 to 1 (i.e., 1:1). In some embodiments, the ratio of milk to media is 1:2, 2:1, 1:3, 3:1, 1:4, or 4:1.

In one embodiment, a method of detecting cancer stem-like cells in a breast milk sample is described comprising: (i) extracting the nucleotides from the sample; (ii) amplifying the nucleotides using primers, and (iii) analyzing the nucleotides in the sample. In some embodiments, the method comprises detecting an increase in p63 expression compared to a control.

The method may include extracting the nucleotides from a breast milk sample; analyzing the nucleotides in the sample for gene expression and breast cancer enriched gene aberrations, and quantifying the expression level of a cancer marker. The method may further include a step of amplifying the nucleotides. The method may further comprise detecting the cancer marker. In some embodiments, the cancer marker is p63.

The disclosed method may indicate cancer when the cancer marker expression level as quantified in the breast milk is increased compared to a control sample. In some embodiments, a control sample is provided by the same individual. For example, the opposite breast than the one tested. In some embodiments, a control value is provided by an average population data set. When the p63 expression is at least about 1.5× higher than the control level, the results may indicate the presence of cancer. When p63 expression is about 1.5×, about 2.0×, about 2.5×, about 3.0× about 3.5×, about 4.0×, about 4.5×, about 5.0×, about 5.5×, about 6.0×, about 6.5×, about 7.0×, about 7.5×, about 8.0×, about 8.5×, about 9.0×, about 9.5×, or about 10.0× higher than the control, the results may indicate the presence of cancer.

In one embodiment, the method further comprises quantifying the expression of a caner marker. In some embodiments, the method comprises comparing the expression levels of a cancer marker identified in the sample with a control. In some embodiments, the cancer marker is p63. In some embodiments, the method is performed close in time to the collection of the sample. In other embodiments, the sample is stored prior to performing the method.

In some embodiments, the sample may include the entire amount of breast milk produced in one sitting from one or both breasts. In another embodiment, the sample may include the entire amount of breast milk produced over a 24 hour period from one or both breasts. In some embodiments, the sample may be collected at different time points of breast milk released during a sitting. For example, one sample may be taken during the initial release of breast milk and a second sample may be taken at the end of release of breast milk.

In some embodiments, the sample is collected using a breast pump and collection container.

In one embodiment, the nucleotides are analyzed for mutations. For example, the mutation may indicate a disease state in the breast milk-derived cells. In one embodiment, the disease is cancer, e.g., the cancer may be breast cancer. In one embodiment, the cancer is a non-metastatic cancer. In one embodiment, the nucleotides are analyzed by PCR, e.g., overlap extension PCR (OE PCR), emulsion PCR (EmPCR), or digital droplet PCR (ddPCR) may be used. In one embodiment, the nucleotides are analyzed for the presence of a tumor marker or a tumor recurrence marker. In some embodiments, the tumor marker is p63. In one embodiment, a DNA elongation method can be employed that transforms small DNA molecules into longer DNA fragments that can be analyzed using standard methods, thus allowing the analysis of more DNA molecules of interest than otherwise possible. In one embodiment, the method further comprises monitoring the patient for cancer progression. In one embodiment, the method further comprises determining if the patient is eligible for a targeted cancer therapy.

As described herein, a genetic marker is a specific sequence of DNA at a known location on a chromosome. Examples of genetic markers may include single polymorphism nucleotides (SNPs) and microsatellites. A genetic marker of susceptibility is a specific change in a person's DNA that makes the person more likely to develop certain diseases such as cancer.

As described herein, a biomarker, or molecular marker, is a biological molecule found in blood, urine, other body fluids such as lymph fluid or breast milk, or tissues that is a sign of a normal or abnormal process, or of a condition or disease. A biomarker may be used to see how well the body responds to a treatment for a disease or condition. Many specific biomarkers have been well characterized and repeatedly shown to correctly predict relevant clinical outcomes across a variety of treatments and populations.

As described herein, tumor markers are substances that are produced by cancer cells or by other cells of the body in response to cancer or certain benign (noncancerous) conditions. Most tumor markers are made by normal cells as well as by cancer cells; however, they are produced at much higher levels in cancerous conditions. These substances can be found in the blood, urine, stool, tumor tissue, or other tissues or bodily fluids of some patients with cancer. In particular here, the substances are found in breast milk. Tumor markers may include, e.g., proteins, DNA, RNA, etc. For example, certain patterns of gene expression and changes to DNA can be used as tumor markers. A tumor recurrence marker is a tumor marker used in monitoring the tumor recurrence in a patient.

The methods described herein may be useful to determine specific molecules, e.g., a tumor marker or a tumor recurrence marker, to predict the risk of tumor relapse after a specific treatment or curative surgery. In one embodiment, ctDNA from a patient sample is analyzed for the presence of a tumor marker, a tumor recurrence marker, a genetic marker, and/or, a biomarker. In one embodiment, the mutant allele fraction of a specific gene is determined. In one embodiment, the heterogeneity of the mutant allele fraction is determined.

In accordance with the invention, “patient” may refer to a human or an animal. Accordingly, the methods and compositions disclosed herein can be used for both human clinical medicine and veterinary applications. Thus, as described herein, a “patient” can be a human or, in the case of veterinary applications, the patient can be a laboratory, an agricultural, a domestic, or a wild animal. In various aspects, the patient can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, a goat, or a wild animal in captivity such as a bear, a panda, a lion, a tiger, a leopard, an elephant, a zebra, a giraffe, a gorilla, a dolphin, or a whale. Exemplary patients include cancer patients, post-operative patients, transplant patients, patients undergoing chemotherapy, immunosuppressed patients, and the like. In one embodiment, the sample is obtained from a patient. In another embodiment, the patient is a female producing breast milk. In some embodiments, the female is pregnant. In some embodiments, the female is postpartum. In another embodiment, the sample is a breast milk sample from a patient. In some embodiments, the sample is collected from a woman who is nursing. The samples can be prepared for testing as described herein.

In various embodiments, the nucleotides (DNA or RNA) are collected from a breast milk sample comprising breast milk-derived cells. In some embodiments, the breast milk-derived cell is a cancer-like stem cell. In some embodiments, the breast milk-derived cell comprises markers to indicate a cancer. In some embodiments, the cancer is a breast cancer. In some embodiments, the breast cancer is HER2+. HER2−, or triple negative breast cancer. In some embodiments, the cancer originated from another source and metastasized into the breast tissue.

In one embodiment, the method is used to extract DNA and detect a disease. In one embodiments, the method is used to extract RNA and detect a disease. In a further embodiment, the disease is cancer. In some embodiments, the cancer comprises a primary tumor. In yet other embodiments, the cancer comprises non-metastatic tumor cells. In yet other embodiments, the cancer comprises metastatic tumor cells.

In one embodiment, detecting the nucleotides in the sample comprises quantifying the copy number of a gene in the nucleotides sample. In one embodiment, detecting the nucleotides in the sample comprises detecting a mutation in the nucleotides sample. In some embodiments, the gene copy number is quantified per ml of sample. The methods described herein can be used to detect or identify specific nucleic acid sequences in a DNA sample. Techniques for isolation of DNA are well-known in the art. Methods for isolating DNA are described in Sambrook et al., “Molecular Cloning: A Laboratory Manual”, 3rd Edition, Cold Spring Harbor Laboratory Press, (2001), incorporated herein by reference.

In one embodiment, a method of analyzing the nucleotides for a mutation is provided including: providing primer(s) and/or probe(s), amplifying the nucleotides, sequencing the nucleotides, and analyzing the sequenced nucleotides for mutations. In various embodiments, the mutation is indicative of a disease, e.g., cancer. In one embodiment, the nucleotides may be analyzed for the presence of a specific mutant allele fraction, a genetic marker, a biomarker, a tumor marker, or a tumor recurrence marker. In one embodiment, the amplification method is a PCR method, such as OE PCR or ddPCR.

In the methods herein described, DNA/mRNA may be detected and/or quantified using any DNA detection method known in the art. In one embodiment, the nucleic acid may be detected using conventional polymerase chain reaction (PCR) methods. In one embodiment, the nucleic acid may be detected using conventional polymerase chain reaction (PCR), quantitative PCR (qPCR), overlap extension PCR (OE PCR), Emulsion PCR (EmPCR), or digital PCR (dPCR). As described herein, PCR techniques may be used to amplify specific, target DNA fragments from low quantities of source DNA or RNA (for example, after a reverse transcription step to produce complementary DNA (cDNA), or detection of small fragment DNAs in a sample). When performing conventional PCR, the final concentration of template is proportional to the starting copy number and the number of amplification cycles. In one embodiment, a given number of reactions is performed on a single sample and the result is an analysis of fragment sizes or, for quantitative real-time PCR (qPCR), the analysis is an estimate of the concentration of the target sequences in the reaction-based on the number of cycles required to reach a quantification cycle (Cq).

For qPCR methods, a fluorescent reporter dye is used as an indirect measure of the amount of nucleic acid present during each amplification cycle. The increase in fluorescent signal is directly proportional to the quantity of exponentially accumulating PCR product molecules (amplicons) produced during the repeating phases of the reaction. Reporter molecules may be categorized as; double-stranded DNA (dsDNA) binding dyes, dyes conjugated to primers, or additional dye-conjugated oligonucleotides, referred to as probes. The use of a dsDNA-binding dye, such as SYBR® Green I, represents the simplest form of detection chemistry. When free in solution or with only single-stranded DNA (ssDNA) present, SYBR Green I dye emits light at low signal intensity. As the PCR progresses and the quantity of dsDNA increases, more dye binds to the amplicons and hence, the signal intensity increases. Alternatively, a probe (or combination of two depending on the detection chemistry) can add a level of detection specificity beyond the dsDNA-binding dye, since it binds to a specific region of the template that is located between the primers. The most commonly used probe format is the Dual-Labeled Probe (DLP; also referred to as a Hydrolysis or TaqMan® Probe). The DLP is an oligonucleotide with a 5′ fluorescent label, e.g., 6-FAM™ and a 3′ quenching molecule, such as one of the dark quenchers e.g., BHQ®1 or OQ™ (see Quantitative PCR and Digital PCR Detection Methods). These probes are designed to hybridize to the template between the two primers and are used in conjunction with a DNA polymerase that has 5′ to 3′ exonuclease activity.

For digital PCR (dPCR), the sample can be diluted and separated into a large number of reaction chambers or partitions. In various embodiments, each partition contains either one copy of the target DNA or no copies of the target DNA. In some embodiments, the partition may contain one or more copies of the target DNA. In some embodiments, the partition may contain two or more copies of the target DNA. The number of reaction chambers or partitions varies between systems, from several thousand to millions. The PCR is then performed in each partition and the amplicon detected using a fluorescent label such that the collected data are a series of positive and negative results.

In one embodiment, the methods described herein may include droplet digital PCR (ddPCR) technology. ddPCR is a method for performing digital PCR that is based on water-oil emulsion droplet technology. For example, a sample is fractionated into thousands of droplets (e.g., 10,000, 15,000, 20,000, 25,000, 30,000, 40,000, or 50,000 droplets, or more depending on the reaction to be performed), and PCR amplification of the template molecules occurs in each individual droplet. The droplets for use in ddPCR are typically nanoliter-sized droplets. ddPCR has a small sample requirement reducing cost and preserving samples.

As described herein, for methods employing ddPCR, the sample(s) may be partitioned into 20,000 nanoliter-sized droplets. This partitioning allows the measurement of thousands of independent amplification events within a single sample. ddPCR technology uses reagents and workflows similar to those used for most standard TaqMan probe-based assays. ddPCR allows the detection of rare DNA target copies, allows the determination of copy number variation, and allows the measurement of gene expression levels with high accuracy and sensitivity. Digital PCR is an end-point PCR method that is used for absolute quantification and for analysis of minority sequences against a background of similar majority sequences, e.g., quantification of somatic mutations. When using this technique, the sample is taken to limiting dilution and the number of positive and negative reactions is used to determine a precise measurement of target concentration. The digital PCR (dPCR) methods may be employed using emulsion beads (e.g., Bio-Rad QX100™ Droplet Digital™ PCR, ddPCR™ system and RainDance Technologies' RainDrop™ instrument). In an alternative format, the reactions may be run on integrated fluidic circuits (chips). These chips have integrated chambers and valves for partitioning samples and reaction reagents (e.g., BioMark™, Fluidigm).

For overlap extension PCR (OE-PCR) the method may be used for DNA elongation, to insert specific mutations at specific points in a sequence, or to splice smaller DNA fragments into a larger polynucleotide. In one embodiment, a method is described for detection of mutations in very small DNA fragments. Small fragment DNA elongation can be accomplished using a variation of overlap extension (OE) PCR. PCR parameters including primer concentration, concentration of PCR additives, PCR annealing temperatures, and temperature ramp speeds are analyzed for contribution to maximizing elongation efficiency. In another embodiment, a method of analyzing the ctDNA for a mutation is provided including: providing primer(s) and/or probe(s), amplifying the ctDNA, sequencing the ctDNA, and analyzing the sequenced ctDNA for mutations. In various embodiments, the mutation is indicative of a disease, e.g., cancer. In one embodiment, the amplification method is a PCR method, such as OE PCR, EmPCR, or ddPCR.

Emulsion PCR (EmPCR) may be used for template amplification, e.g., in multiple NGS-based sequencing platforms. The basic principle of emPCR is dilution and compartmentalization of template molecules in water droplets in a water-in-oil emulsion. Ideally, the dilution is to a degree where each droplet contains a single template molecule and functions as a micro-PCR reaction. As described herein, emulsion PCR can overcome possible OE PCR bias for elongation of ultra-low frequency mutations. Elongation efficiency and false positive mutation rates may be analyzed to determine optimal PCR conditions, utilizing in vitro systems of varying mutant and wildtype DNA fragment sizes and ratios, and modeling human breast milk, which contains DNA of differing fragment lengths.

Techniques for isolation of DNA and RNA are well-known in the art. In one embodiment, cells may be ruptured by using a detergent or a solvent, such as phenolchloroform. In another embodiment, cells remain intact and cell-free DNA may be extracted. DNA may be separated from other components in the sample by physical methods including, but not limited to, centrifugation, pressure techniques, or by using a substance with affinity for DNA, such as, for example, silica beads. After sufficient washing, the isolated DNA may be suspended in either water or a buffer. In other embodiments, commercial kits are available, such as Quiagen™, Nuclisensm™, and Wizard™ (Promega), and Promegam™. Methods for isolating DNA are described in Sambrook et al., “Molecular Cloning: A Laboratory Manual”, 3rd Edition, Cold Spring Harbor Laboratory Press, (2001), incorporated herein by reference.

In various embodiments described herein, primers and/or probes are used for amplification of the target DNA or RNA are oligonucleotides from about ten to about one hundred, more typically from about ten to about thirty or about twenty to about twenty-five base pairs long, but any suitable sequence length can be used. In illustrative embodiments, the primers and probes may be double-stranded or single-stranded, but the primers and probes are typically single-stranded. The primers and probes described herein are capable of specific hybridization, under appropriate hybridization conditions (e.g., appropriate buffer, ionic strength, temperature, formamide, or MgCl₂ concentrations), to a region of the target DNA. The primers and probes described herein may be designed based on having a melting temperature within a certain range, and substantial complementarity to the target DNA. Methods for the design of primers and probes are described in Sambrook et al., “Molecular Cloning: A Laboratory Manual”, 3rd Edition, Cold Spring Harbor Laboratory Press, (2001), incorporated herein by reference.

Also within the scope of the invention are nucleic acids complementary to the probes and primers described herein, and those that hybridize to the nucleic acids described herein or those that hybridize to their complements under highly stringent conditions. In accordance with the invention “highly stringent conditions” means hybridization at 65° C. in 5×SSPE and 50% formamide, and washing at 65° C. in 0.5×SSPE. Conditions for low stringency and moderately stringent hybridization are described in Sambrook et al., “Molecular Cloning: A Laboratory Manual”, 3rd Edition, Cold Spring Harbor Laboratory Press, (2001), incorporated herein by reference. In some illustrative aspects, hybridization occurs along the full-length of the nucleic acid.

In some embodiments, also included are nucleic acid molecules having about 60%, about 70%, about 75%, about 80%, about 85%, about 90%, about 95%, at least 96%, at least 97%, at least 98%, or at least 99% homology to the probes and primers described herein. Determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software. In some embodiments, the percent identity can be determined along the full-length of the nucleic acid. As used herein, the term “complementary” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

Techniques for synthesizing the probes and primers described herein are well-known in the art and include chemical syntheses and recombinant methods. Such techniques are described in Sambrook et al., “Molecular Cloning: A Laboratory Manual”, 3rd Edition, Cold Spring Harbor Laboratory Press, (2001), incorporated herein by reference. Primers and probes can also be made commercially (e.g., CytoMol, Sunnyvale, CA or Integrated DNA Technologies, Skokie, IL). Techniques for purifying or isolating the probes and primers described herein are well-known in the art. Such techniques are described in Sambrook et al., “Molecular Cloning: A Laboratory Manual”, 3rd Edition, Cold Spring Harbor Laboratory Press, (2001), incorporated herein by reference. The primers and probes described herein can be analyzed by techniques known in the art, such as restriction enzyme analysis or sequencing, to determine if the sequence of the primers and probes is correct.

The following examples are exemplary embodiments of the disclosure. One of ordinary skill in the art will understand that slight variations or substitutions may be made to achieve the same results. Those slight variations and substitutions are considered a part of the disclosure herein.

### EXAMPLES

### Example 1

### Breast Milk-Derived Cell Culture and Donor Information

Breast milk was collected in sterile cups and cultured within an hour of donation directly on 60-mm tissue culture plates pre-coated with conditioned media from 804G cells overnight in 50% culture media along with ROCK, TGFβ and BMP inhibitors and Cipro. In total, breast milk from 11 women were cultured and characterized. Five of them (all Caucasian) completed a questionnaire, which requested information including the number of pregnancies, the duration of breast-feeding, smoking, and contraceptive use. Time of milk collection varied between 1-5 months after delivery. Four women were nursing from their first pregnancy and one was nursing from her second pregnancy. None of the women had a breast cancer diagnosis or known BRCA mutations. Two women had a family history of breast cancer. All of the women had used birth control pills. There were no discernable differences between donors and the age range of donors was 29-37 years.

Cell culture plates with breast milk were washed extensively with PBS the next day and cells were passaged once plates became confluent. We usually observed faster growth of cells from milk from the breasts with abnormalities. Flow cytometry characterization and antibodies were used.

### Example 2

### Mammosphere Culture, Cell Proliferation Assays, and Drug Sensitivity Studies

Mammosphere assays were performed using MammoCult media (Stem Cell Technologies) as described previously. Cell proliferation rates were measured using Bromodeoxyuridine ELISA by plating 1000 cells in 96 well plates precoated with conditioned media from 804G cells overnight. Sensitivity to drugs was measured after five days of treatment with one media and drug exchange. HDAC6 inhibitor CAY10603 (#S7596), EZH2 inhibitor GSK126 (#S7061), and the BET bromodomain inhibitor JQ1 (#S7110) were purchased from Selleckchem.com. We used an unpaired t test (Graphpad.com) to determine statistical differences in proliferation rate and data from technical replicates are presented although drug sensitivity studies were performed thrice.

### Example 3

### RNA and DNA Preparation for Sequencing

RNA from biologic triplicates was prepared using the RNAeasy kit from Qiagen and. Total DNA was prepared using the Qiagen DNA mini kit (Cat #51304). The primers were purchased from Applied Biosciences.
FOXA1: Assay ID: HS04187555_ml and catalog #: 4331182—the assay spans exons 1 and 2 boundary.
GATA3: Assay ID: H500231122_ml and catalog #: 4331182—the assay spans exon 2-3 boundary.
ESR1: Assay ID: HS00174860 and catalog #: 4331182—assay spans exon 3-4 boundary.

### Example 4

### Sequence Alignment and Gene Counts for RNA-Seq

RNA sequencing was performed. The sequencing data were first assessed using FastQC (v.0.11.5, Babraham Bioinformatics, Cambridge, UK) for quality control. All sequenced libraries were mapped to the human genome (UCSC hg38) using the STAR RNA-seq aligner (v.2.5) with the following parameter: “--outSAMmapqUnique 60”. The read distribution across the genome was assessed using bamutils (from ngsutils v.0.5.9). Uniquely mapped sequencing reads were assigned to hg38 refGene genes using featureCounts (subread v.1.5.1) with the following parameters: “-s 2-p-Q 10”. Each sample was analyzed independently and genes with read count per million (CPM)<0.5 in more than 3 replicate samples were removed from the comparisons. The data was normalized using the TMM (trimmed mean of M values) method. Multi-dimensional scaling analysis was done with limma (v.3.38.3). Differential expression analyses were performed using edgeR (v.3.12.1). The false discovery rate (FDR) was computed from p-values using the Benjamini-Hochberg procedure.

### Example 5

### DNA Sequencing, Alignment, and Mutation Detection

Whole genome sequencing libraries were generated with Illumina Nextera DNA Flex Library Prep Kit according to the manufacturer's instruction. The libraries were sequenced on Illumina NovaSeq 6000 S4 flow cell with 150 bp paired-end reads. Breast milk-derived cell DNA and blood samples were targeted for ˜80× and 40× coverage, respectively. All sequence data will be submitted to NCBI dbGaP server.

The paired-end sequence reads were first processed to remove Illumine adapter sequences and low-quality base calls with Trim Galore (http://www.bioinformatics.babraham.ac.uk/projects/trim_galore/). The resulting high-quality reads were aligned to human reference genome hg38 using BWA-MEM and BWAKIT (v0.7.15). Sentieon version 201911 (Sentieon, Inc, https://www.sentieon.com/) was applied in the following step to identify somatic variants. The sentieon analysis included duplicated reads marking, indel realignment, base quality recalibration (BQSR), and co-realignment of tumor and normal samples. Quality metrics including alignment stats, whole genome sequence metrics and insert size metrics were generated during the process. Somatic variants were detected with the Sentieon TNSeq algorithm. The variants passed filters were next functionally annotated with ANNOVAR using various databases.

### Example 6

### Copy Number Variation (CNV) Measurement Using nanoString

We used nCounter® v2 Cancer Copy number assay (115000112: XT_PCN_HuV2_Cancercopy-CSO from Nanostring) to determine CNVs in 87 genes commonly deleted or amplified in cancer. The assay was done as per manufacturer recommendations using 300 ngs of genomic DNA. Values 1.6-2.4 are considered normal, 2.6-3.4 considered amplification, whereas 0.4 to 1.4 are considered deletions. For M1 and M2, DNA from the right breast milk-derived cells was compared to DNA from the left breast milk-derived cells, whereas for M10, DNA from the left breast milk-derived cells was compared to DNA from the right breast milk-derived cells. Thus, controls for the assay is from the same individual. Raw numbers of triplicate assays are presented in Tables 1, 2, and 3.

TABLE 1
shows the copy number variation in 87 genes in M1-right breast milk
derived cells. DNA from the corresponding contralateral breast milk-
derived cells were used as controls to determine the number. Values
1.6 to 2.4 are considered to be normal without genomic alterations.
Lower number is deletion and higher number amplification.

| | Copy Number | | | | | |
|---|---|---|---|---|---|---|
| Target | 1 | 2 | 3 | Average | stdev | std error |
| TP73 | 2.23 | 2.31 | 1.84 | 2.127 | 0.251 | 0.101 |
| MYCL1 | 1.79 | 1.84 | 2.13 | 1.920 | 0.184 | 0.073 |
| CDKN2C | 2.95 | 1.67 | 2.05 | 2.223 | 0.657 | 0.263 |
| JUN | 2.03 | 1.99 | 1.99 | 2.003 | 0.023 | 0.009 |
| MAG13 | 2.26 | 1.74 | 2.12 | 2.040 | 0.269 | 0.108 |
| REG4 | 2.12 | 1.9 | 1.82 | 1.947 | 0.155 | 0.062 |
| MCL1 | 2.2 | 2.35 | 2.21 | 2.253 | 0.084 | 0.034 |
| MDM4 | 2.15 | 2.24 | 2.14 | 2.177 | 0.055 | 0.022 |
| AKT3 | 1.83 | 2.11 | 2.14 | 2.027 | 0.171 | 0.068 |
| MYCN | 2.2 | 1.91 | 2.07 | 2.060 | 0.145 | 0.058 |
| REL | 2.2 | 2.25 | 2.19 | 2.213 | 0.032 | 0.013 |
| FHIT | 1.96 | 2.05 | 1.78 | 1.930 | 0.137 | 0.055 |
| MITF | 2.2 | 1.75 | 2.13 | 2.027 | 0.242 | 0.097 |
| PRKC1 | 2.36 | 2.17 | 1.88 | 2.137 | 0.242 | 0.097 |
| PIK3CA | 2.12 | 1.86 | 2.5 | 2.160 | 0.322 | 0.129 |
| DCUN1D1 | 1.92 | 2.01 | 1.95 | 1.960 | 0.046 | 0.018 |
| PDGFRA | 2.18 | 1.96 | 1.82 | 1.987 | 0.181 | 0.073 |
| KIT | 2.09 | 1.99 | 1.92 | 2.000 | 0.085 | 0.034 |
| KDR | 1.82 | 2.23 | 2.26 | 2.103 | 0.246 | 0.098 |
| TERT | 1.95 | 1.8 | 1.73 | 1.827 | 0.112 | 0.045 |
| SKP2 | 1.74 | 2.01 | 2.44 | 2.063 | 0.353 | 0.141 |
| PDE4D | 1.85 | 1.97 | 2.03 | 1.950 | 0.092 | 0.037 |
| APC | 1.88 | 2.1 | 2.25 | 2.077 | 0.186 | 0.074 |
| E2F3 | 2.13 | 2.22 | 2.23 | 2.193 | 0.055 | 0.022 |
| CDKN1A | 1.81 | 2.32 | 1.99 | 2.040 | 0.259 | 0.103 |
| VEGFA | 2.05 | 2.37 | 1.85 | 2.090 | 0.262 | 0.105 |
| MYB | 2.06 | 2.11 | 1.77 | 1.980 | 0.184 | 0.073 |
| MAP3K5 | 2.22 | 1.9 | 1.81 | 1.977 | 0.215 | 0.086 |
| PARK2 | 1.99 | 1.96 | 2 | 1.983 | 0.021 | 0.008 |
| EGFR | 2 | 1.7 | 2.27 | 1.990 | 0.285 | 0.114 |
| CDK6 | 1.9 | 2.04 | 1.95 | 1.963 | 0.071 | 0.028 |
| MET | 2.09 | 2.73 | 1.99 | 2.270 | 0.401 | 0.161 |
| SHH | 2.2 | 2.13 | 1.96 | 2.097 | 0.123 | 0.049 |
| CSMD1 | 1.79 | 2.05 | 2.03 | 1.957 | 0.145 | 0.058 |
| WHSC1L1 | 1.78 | 1.83 | 2.1 | 1.903 | 0.172 | 0.069 |
| FGFR1 | 1.83 | 2.21 | 2.08 | 2.040 | 0.193 | 0.077 |
| C8orf4 | 1.97 | 1.73 | | 1.850 | 0.170 | 0.068 |
| YWHAZ | 1.86 | 1.9 | 1.76 | 1.840 | 0.072 | 0.029 |
| MYC | 1.99 | 2.1 | 1.93 | 2.007 | 0.086 | 0.034 |
| PTPRD | 2.04 | 2.25 | 1.82 | 2.037 | 0.215 | 0.086 |
| CDKN2A | 2.34 | 1.79 | 2.29 | 2.140 | 0.304 | 0.122 |
| MELK | 2.36 | 2.09 | 1.98 | 2.143 | 0.196 | 0.078 |
| TRAF2 | 2.59 | 1.84 | 2.28 | 2.237 | 0.377 | 0.151 |
| PTEN | 2.18 | 2 | 2 | 2.060 | 0.104 | 0.042 |
| WT1 | 2.12 | 1.97 | 1.78 | 1.957 | 0.170 | 0.068 |
| CCND1 | 1.86 | 1.99 | 2.12 | 1.990 | 0.130 | 0.052 |
| ORAOV1 | 1.8 | 2.2 | 1.86 | 1.953 | 0.216 | 0.086 |
| FADD | 2.39 | 1.8 | 2.05 | 2.080 | 0.296 | 0.118 |
| GAB2 | 1.86 | 1.79 | 1.91 | 1.853 | 0.060 | 0.024 |
| YAP1 | 1.97 | 1.8 | 2.06 | 1.943 | 0.132 | 0.053 |
| BIRC2 | 2.2 | 1.94 | 2.04 | 2.060 | 0.131 | 0.052 |
| CCND2 | 1.71 | 1.83 | 2.43 | 1.990 | 0.386 | 0.154 |
| KRAS | 1.92 | 1.72 | 2.07 | 1.903 | 0.176 | 0.070 |
| CDK4 | 2.1 | 2.11 | 2 | 2.070 | 0.061 | 0.024 |
| HMGA2 | 1.91 | 2.21 | 2.02 | 2.047 | 0.152 | 0.061 |
| DYRK2 | 1.72 | 2.1 | 1.89 | 1.903 | 0.190 | 0.076 |
| MDM2 | 2.18 | 2.01 | 2.07 | 2.087 | 0.086 | 0.034 |
| BRCA2 | 2.52 | 1.75 | 2.01 | 2.093 | 0.392 | 0.157 |
| FOXO1 | 1.93 | 1.82 | 1.87 | 1.873 | 0.055 | 0.022 |
| RB1 | 2 | 2 | 1.83 | 1.943 | 0.098 | 0.039 |
| GPC5 | 2.1 | 1.98 | 1.79 | 1.957 | 0.156 | 0.063 |
| IRS2 | 1.89 | 1.99 | 1.91 | 1.930 | 0.053 | 0.021 |
| BCL2L2 | 2.22 | 1.71 | 2.46 | 2.130 | 0.383 | 0.153 |
| NKX2-1 | 2.21 | 1.91 | | 2.060 | 0.212 | 0.085 |
| NKX2-8 | 2.33 | 1.68 | | 2.005 | 0.460 | 0.184 |
| PAX9 | 1.77 | 1.88 | 1.95 | 1.867 | 0.091 | 0.036 |
| IGF1R | 2.12 | 2.18 | 2.08 | 2.127 | 0.050 | 0.020 |
| TP53 | 2.09 | 2.16 | 2.27 | 2.173 | 0.091 | 0.036 |
| MAP2K4 | 2 | 2.17 | 2.07 | 2.080 | 0.085 | 0.034 |
| MAPK7 | 2.21 | 2.21 | 2.79 | 2.403 | 0.335 | 0.134 |
| NF1 | 1.93 | 2.21 | 1.9 | 2.013 | 0.171 | 0.068 |
| ERBB2 | 2.45 | 2 | 2.08 | 2.177 | 0.240 | 0.096 |
| BRCA1 | 1.86 | 1.97 | 2 | 1.943 | 0.074 | 0.029 |
| RPS6KB1 | 2.21 | 1.88 | 1.97 | 2.020 | 0.171 | 0.068 |
| GRB2 | 2.08 | 1.82 | 1.99 | 1.963 | 0.132 | 0.053 |
| ITGB4 | 2.29 | 2.1 | 2.11 | 2.167 | 0.107 | 0.043 |
| DCC | 1.65 | 2.2 | 1.87 | 1.907 | 0.277 | 0.111 |
| CCNE1 | 2.09 | 1.89 | 1.82 | 1.933 | 0.140 | 0.056 |
| AKT2 | 2 | 2.19 | 2.43 | 2.207 | 0.215 | 0.086 |
| BBC3 | 2.19 | 2.03 | 2.02 | 2.080 | 0.095 | 0.038 |
| BCL2L1 | 2.06 | 2.48 | 1.88 | 2.140 | 0.308 | 0.123 |
| NCOA3 | 1.97 | 2.15 | 1.93 | 2.017 | 0.117 | 0.047 |
| ZNF217 | 2.29 | 1.93 | 2.17 | 2.130 | 0.183 | 0.073 |
| AURKA | 1.93 | 1.89 | 2.21 | 2.010 | 0.174 | 0.070 |
| EEF1A2 | 2.19 | 2.03 | 1.84 | 2.020 | 0.175 | 0.070 |
| CRKL | 2.02 | 2.26 | 2.05 | 2.110 | 0.131 | 0.052 |

TABLE 2
shows the copy number variation in 87 genes in M2-right breast milk
derived cells. DNA from the corresponding contralateral breast milk-
derived cells were used as controls to determine the number. Values
1.6 to 2.4 are considered to be normal without genomic alterations.
Lower number is deletion and higher number amplification.

| | Copy Number | | | | | |
|---|---|---|---|---|---|---|
| Target | 1 | 2 | 3 | Average | stdev | std error |
| TP73 | 2.43 | 1.93 | 2.59 | 2.317 | 0.344287 | 0.138 |
| MYCL1 | 2.29 | 2.24 | 2.22 | 2.250 | 0.036056 | 0.014 |
| CDKN2C | 2.69 | 2.3 | 2.7 | 2.563 | 0.228108 | 0.091 |
| JUN | 2.17 | 2.37 | 2.55 | 2.363 | 0.190088 | 0.076 |
| MAG13 | 2.37 | 2.25 | 2.23 | 2.283 | 0.075719 | 0.030 |
| REG4 | 2.1 | 2.31 | 2.57 | 2.327 | 0.235443 | 0.094 |
| MCL1 | 2.11 | 1.86 | 2.65 | 2.207 | 0.403774 | 0.162 |
| MDM4 | 2.5 | 2.33 | 1.96 | 2.263 | 0.276104 | 0.110 |
| AKT3 | 2.24 | 1.97 | 2.13 | 2.113 | 0.135769 | 0.054 |
| MYCN | 2.78 | 2.17 | 2.38 | 2.443 | 0.309892 | 0.124 |
| REL | 2.77 | 1.97 | 2.81 | 2.517 | 0.473849 | 0.190 |
| FHIT | 1.86 | 1.7 | 1.8 | 1.787 | 0.080829 | 0.032 |
| MITF | 2.01 | 1.84 | 1.99 | 1.947 | 0.092916 | 0.037 |
| PRKC1 | 2.01 | 1.85 | 2.65 | 2.170 | 0.42332 | 0.169 |
| PIK3CA | 2.26 | 2.45 | 2.56 | 2.423 | 0.151767 | 0.061 |
| DCUN1D1 | 2.14 | 2.34 | 1.95 | 2.143 | 0.195021 | 0.078 |
| PDGFRA | 1.87 | 2.04 | 2.04 | 1.983 | 0.09815 | 0.039 |
| KIT | 1.86 | 2.49 | 2 | 2.117 | 0.330807 | 0.132 |
| KDR | 2.45 | 2.42 | 1.95 | 2.273 | 0.280416 | 0.112 |
| TERT | 2.65 | 2.08 | 1.93 | 2.220 | 0.379868 | 0.152 |
| SKP2 | 2.25 | 2.38 | 2.05 | 2.227 | 0.166233 | 0.066 |
| PDE4D | 2.54 | 2.19 | 2.26 | 2.330 | 0.185203 | 0.074 |
| APC | 1.85 | 2.1 | 1.82 | 1.923 | 0.153731 | 0.061 |
| E2F3 | 2.3 | 2.19 | 2.12 | 2.203 | 0.090738 | 0.036 |
| CDKN1A | 2.75 | 2.03 | 2.27 | 2.350 | 0.366606 | 0.147 |
| VEGFA | 2.3 | 2.25 | 2.1 | 2.217 | 0.104083 | 0.042 |
| MYB | 2.67 | 2.35 | 2.13 | 2.383 | 0.271539 | 0.109 |
| MAP3K5 | 2.33 | 2.26 | 2.36 | 2.317 | 0.051316 | 0.021 |
| PARK2 | 2.09 | 2.44 | 2.43 | 2.320 | 0.199249 | 0.080 |
| EGFR | 1.68 | 2.18 | 2.17 | 2.010 | 0.285832 | 0.114 |
| CDK6 | 2.38 | 2.39 | 2.35 | 2.373 | 0.020817 | 0.008 |
| MET | 2.4 | 2.15 | 2.36 | 2.303 | 0.134288 | 0.054 |
| SHH | 2.36 | 2.52 | 1.93 | 2.270 | 0.305123 | 0.122 |
| CSMD1 | 2.77 | 1.86 | 2.43 | 2.353 | 0.459819 | 0.184 |
| WHSC1L1 | 2.1 | 1.77 | 2.21 | 2.027 | 0.228983 | 0.092 |
| FGFR1 | 2.55 | 2.34 | 2.19 | 2.360 | 0.180831 | 0.072 |
| C8orf4 | 2.24 | 2.48 | | 2.360 | 0.169706 | 0.068 |
| YWHAZ | 2.02 | 1.93 | 2.33 | 2.093 | 0.209841 | 0.084 |
| MYC | 2.81 | 2.33 | 2.24 | 2.460 | 0.306431 | 0.123 |
| PTPRD | 1.96 | 2.3 | 2 | 2.087 | 0.185831 | 0.074 |
| CDKN2A | 1.85 | 2.69 | 2.06 | 2.200 | 0.43715 | 0.175 |
| MELK | 2.02 | 2.09 | 2.08 | 2.063 | 0.037859 | 0.015 |
| TRAF2 | 1.94 | 1.76 | 2.46 | 2.053 | 0.363501 | 0.145 |
| PTEN | 2.45 | 2.56 | 2.73 | 2.580 | 0.141067 | 0.056 |
| WT1 | 2.31 | 2.17 | 2.38 | 2.287 | 0.106927 | 0.043 |
| CCND1 | 1.98 | 1.9 | 1.95 | 1.943 | 0.040415 | 0.016 |
| ORAOV1 | 2.47 | 2.04 | 2.35 | 2.287 | 0.221886 | 0.089 |
| FADD | 2.48 | 1.97 | 1.92 | 2.123 | 0.309892 | 0.124 |
| GAB2 | 1.97 | 2.66 | 2.14 | 2.257 | 0.35949 | 0.144 |
| YAP1 | 2.09 | 2.19 | 2.11 | 2.130 | 0.052915 | 0.021 |
| BIRC2 | 2.03 | 2.3 | 2.35 | 2.227 | 0.172143 | 0.069 |
| CCND2 | 2.07 | 2.07 | 2.94 | 2.360 | 0.502295 | 0.201 |
| KRAS | 2.22 | 2.41 | 2.39 | 2.340 | 0.104403 | 0.042 |
| CDK4 | 1.61 | 1.91 | 2.44 | 1.987 | 0.420278 | 0.168 |
| HMGA2 | 2.41 | 2.1 | 2.2 | 2.237 | 0.158219 | 0.063 |
| DYRK2 | 2.17 | 2.33 | 2.12 | 2.207 | 0.109697 | 0.044 |
| MDM2 | 2.55 | 2.35 | 2.11 | 2.337 | 0.220303 | 0.088 |
| BRCA2 | 1.52 | 2.22 | 2.31 | 2.017 | 0.432474 | 0.173 |
| FOXO1 | 2.05 | 2.11 | 2.19 | 2.117 | 0.070238 | 0.028 |
| RB1 | 1.97 | 2.26 | 2.73 | 2.320 | 0.383536 | 0.153 |
| GPC5 | 2.2 | 2.07 | 2.15 | 2.140 | 0.065574 | 0.026 |
| IRS2 | 2.13 | 2.24 | 2.8 | 2.390 | 0.359305 | 0.144 |
| BCL2L2 | 1.58 | 1.9 | 1.91 | 1.797 | 0.187705 | 0.075 |
| NKX2-1 | 2.23 | 2.19 | | 2.210 | 0.028284 | 0.011 |
| NKX2-8 | 2.12 | 1.62 | | 1.870 | 0.353553 | 0.141 |
| PAX9 | 2.4 | 2.92 | 2.16 | 2.493 | 0.388501 | 0.155 |
| IGFIR | 2.16 | 2.13 | 2.08 | 2.123 | 0.040415 | 0.016 |
| TP53 | 2.06 | 2.02 | 1.44 | 1.840 | 0.346987 | 0.139 |
| MAP2K4 | 2.19 | 2.41 | 2.32 | 2.307 | 0.110604 | 0.044 |
| MAPK7 | 2.43 | 2.04 | 2.4 | 2.290 | 0.217025 | 0.087 |
| NF1 | 2.74 | 2.24 | 2.38 | 2.453 | 0.257941 | 0.103 |
| ERBB2 | 2.35 | 2.23 | 2.25 | 2.277 | 0.064291 | 0.026 |
| BRCA1 | 2.38 | 1.79 | 2.08 | 2.083 | 0.295014 | 0.118 |
| RPS6KB1 | 2 | 2.19 | 2.41 | 2.200 | 0.205183 | 0.082 |
| GRB2 | 2.08 | 2.71 | 2.24 | 2.343 | 0.327465 | 0.131 |
| ITGB4 | 2.1 | 2.16 | 1.77 | 2.010 | 0.21 | 0.084 |
| DCC | 2.03 | 2.02 | 2.49 | 2.180 | 0.268514 | 0.107 |
| CCNE1 | 2.19 | 1.92 | 1.94 | 2.017 | 0.150444 | 0.060 |
| AKT2 | 2.39 | 1.83 | 2.06 | 2.093 | 0.281484 | 0.113 |
| BBC3 | 1.79 | 1.81 | 2.17 | 1.923 | 0.213854 | 0.086 |
| BCL2L1 | 2.39 | 2.61 | 2.32 | 2.440 | 0.151327 | 0.061 |
| NCOA3 | 2.18 | 2.57 | 1.91 | 2.220 | 0.331813 | 0.133 |
| ZNF217 | 2.38 | 2.14 | 2.19 | 2.237 | 0.126623 | 0.051 |
| AURKA | 2.01 | 2.53 | 2.21 | 2.250 | 0.262298 | 0.105 |
| EEF1A2 | 2.2 | 1.83 | 1.66 | 1.897 | 0.276104 | 0.110 |
| CRKL | 2 | 2.41 | 2.37 | 2.260 | 0.226053 | 0.090 |

TABLE 3
shows the copy number variation in 87 genes in M10-left breast milk
derived cells. DNA from the corresponding contralateral breast milk-
derived cells were used as controls to determine the number. Values
1.6 to 2.4 are considered to be normal without genomic alterations.
Lower number is deletion and higher number amplification.

| | Copy Number | | | | | |
|---|---|---|---|---|---|---|
| Target | 1 | 2 | 3 | Average | stdev | std error |
| TP73 | 1.98 | 2.05 | 1.92 | 1.983 | 0.065064 | 0.0260 |
| MYCL1 | 2.04 | 2.03 | 2.09 | 2.053 | 0.032146 | 0.0129 |
| CDKN2C | 1.71 | 2.02 | 2.09 | 1.940 | 0.202237 | 0.0809 |
| JUN | 2.09 | 2.29 | 2.48 | 2.287 | 0.195021 | 0.0780 |
| MAG13 | 2.12 | 1.89 | 2.22 | 2.077 | 0.169214 | 0.0677 |
| REG4 | 1.93 | 2.35 | 2.07 | 2.117 | 0.213854 | 0.0855 |
| MCL1 | 2.42 | 1.82 | 2.01 | 2.083 | 0.306649 | 0.1227 |
| MDM4 | 2.72 | 2.22 | 2.04 | 2.327 | 0.352326 | 0.1409 |
| AKT3 | 2.03 | 1.94 | 1.97 | 1.980 | 0.045826 | 0.0183 |
| MYCN | 1.95 | 2.01 | 1.99 | 1.983 | 0.030551 | 0.0122 |
| REL | 1.56 | 1.84 | 1.21 | 1.537 | 0.315647 | 0.1263 |
| FHIT | 1.94 | 1.84 | 2.14 | 1.973 | 0.152753 | 0.0611 |
| MITF | 2.16 | 2.32 | 2.21 | 2.230 | 0.081854 | 0.0327 |
| PRKC1 | 2.09 | 2.02 | 1.88 | 1.997 | 0.106927 | 0.0428 |
| PIK3CA | 1.96 | 2.02 | 1.67 | 1.883 | 0.187172 | 0.0749 |
| DCUN1D1 | 1.85 | 2.29 | 1.67 | 1.937 | 0.318957 | 0.1276 |
| PDGFRA | 2.09 | 1.86 | 1.82 | 1.923 | 0.145717 | 0.0583 |
| KIT | 2.42 | 2.36 | 2.12 | 2.300 | 0.158745 | 0.0635 |
| KDR | 2.06 | 1.93 | 2.22 | 2.070 | 0.145258 | 0.0581 |
| TERT | 2.01 | 1.58 | 1.88 | 1.823 | 0.22053 | 0.0882 |
| SKP2 | 2.23 | 1.9 | 1.75 | 1.960 | 0.245561 | 0.0982 |
| PDE4D | 2.27 | 2.15 | 1.97 | 2.130 | 0.150997 | 0.0604 |
| APC | 2.34 | 1.57 | 1.82 | 1.910 | 0.39281 | 0.1571 |
| E2F3 | 1.69 | 2.08 | 2.39 | 2.053 | 0.350761 | 0.1403 |
| CDKN1A | 1.75 | 1.54 | 2.63 | 1.973 | 0.578302 | 0.2313 |
| VEGFA | 2.19 | 1.91 | 2 | 2.033 | 0.142945 | 0.0572 |
| MYB | 1.77 | 2.12 | 2.46 | 2.117 | 0.345012 | 0.1380 |
| MAP3K5 | 1.89 | 2.06 | 2.27 | 2.073 | 0.190351 | 0.0761 |
| PARK2 | 2.58 | 2.1 | 1.99 | 2.223 | 0.313741 | 0.1255 |
| EGFR | 1.8 | 2.15 | 2.01 | 1.987 | 0.176163 | 0.0705 |
| CDK6 | 2.02 | 2.21 | 1.98 | 2.070 | 0.122882 | 0.0492 |
| MET | 1.73 | 1.16 | 2.14 | 1.677 | 0.492172 | 0.1969 |
| SHH | 2.05 | 1.6 | 1.68 | 1.777 | 0.240069 | 0.0960 |
| CSMD1 | 1.76 | 2.01 | 1.85 | 1.873 | 0.126623 | 0.0506 |
| WHSC1L1 | 2.27 | 2 | 2.05 | 2.107 | 0.143643 | 0.0575 |
| FGFR1 | 2.09 | 2.04 | 1.92 | 2.017 | 0.087369 | 0.0349 |
| C8orf4 | 1.98 | 2 | | 1.990 | 0.014142 | 0.0057 |
| YWHAZ | 1.92 | 2.65 | 2.11 | 2.227 | 0.378726 | 0.1515 |
| MYC | 1.91 | 1.74 | 2.22 | 1.957 | 0.243379 | 0.0974 |
| PTPRD | 1.99 | 1.6 | 2.17 | 1.920 | 0.291376 | 0.1166 |
| CDKN2A | 1.84 | 1.99 | 2.55 | 2.127 | 0.37421 | 0.1497 |
| MELK | 2.07 | 1.69 | 2.2 | 1.987 | 0.265016 | 0.1060 |
| TRAF2 | 2.19 | 1.74 | 1.93 | 1.953 | 0.225906 | 0.0904 |
| PTEN | 1.78 | 1.81 | 1.57 | 1.720 | 0.130767 | 0.0523 |
| WT1 | 2.21 | 2.03 | 2.04 | 2.093 | 0.10116 | 0.0405 |
| CCND1 | 1.9 | 1.8 | 2.3 | 2.000 | 0.264575 | 0.1058 |
| ORAOV1 | 2.2 | 2 | 2.38 | 2.193 | 0.190088 | 0.0760 |
| FADD | 1.88 | 2.03 | 2 | 1.970 | 0.079373 | 0.0317 |
| GAB2 | 1.99 | 2.22 | 2.01 | 2.073 | 0.12741 | 0.0510 |
| YAP1 | 2.08 | 2.21 | 1.95 | 2.080 | 0.13 | 0.0520 |
| BIRC2 | 2.08 | 1.81 | 2.16 | 2.017 | 0.183394 | 0.0734 |
| CCND2 | 2.13 | 2.27 | 1.52 | 1.973 | 0.39879 | 0.1595 |
| KRAS | 1.92 | 1.59 | 2.09 | 1.867 | 0.254231 | 0.1017 |
| CDK4 | 2.17 | 1.81 | 1.89 | 1.957 | 0.189033 | 0.0756 |
| HMGA2 | 2.04 | 1.86 | 2.08 | 1.993 | 0.117189 | 0.0469 |
| DYRK2 | 1.79 | 1.9 | 1.96 | 1.883 | 0.086217 | 0.0345 |
| MDM2 | 2.12 | 1.79 | 2 | 1.970 | 0.167033 | 0.0668 |
| BRCA2 | 1.97 | 2 | 2.07 | 2.013 | 0.051316 | 0.0205 |
| FOXO1 | 1.92 | 2.02 | 2.13 | 2.023 | 0.10504 | 0.0420 |
| RB1 | 2.23 | 2.75 | 1.99 | 2.323 | 0.388501 | 0.1554 |
| GPC5 | 2.01 | 2.25 | 2.11 | 2.123 | 0.120554 | 0.0482 |
| IRS2 | 1.79 | 1.94 | 1.66 | 1.797 | 0.140119 | 0.0560 |
| BCL2L2 | 1.9 | 2.25 | 1.99 | 2.047 | 0.181751 | 0.0727 |
| NKX2-1 | 2.03 | 1.86 | | 1.945 | 0.120208 | 0.0481 |
| NKX2-8 | 1.73 | 1.99 | | 1.860 | 0.183848 | 0.0735 |
| PAX9 | 1.53 | 1.57 | 1.9 | 1.667 | 0.20306 | 0.0812 |
| IGF1R | 1.94 | 1.54 | 1.82 | 1.767 | 0.205264 | 0.0821 |
| TP53 | 2.22 | 2.01 | 1.45 | 1.893 | 0.398037 | 0.1592 |
| MAP2K4 | 1.84 | 2.41 | 2.11 | 2.120 | 0.285132 | 0.1141 |
| MAPK7 | 1.99 | 2.27 | 1.72 | 1.993 | 0.275015 | 0.1100 |
| NF1 | 1.61 | 1.73 | 2.29 | 1.877 | 0.362951 | 0.1452 |
| ERBB2 | 2.26 | 1.85 | 1.92 | 2.010 | 0.219317 | 0.0877 |
| BRCA1 | 1.83 | 1.99 | 2.11 | 1.977 | 0.140475 | 0.0562 |
| RPS6KB1 | 1.67 | 2.08 | 2.66 | 2.137 | 0.497427 | 0.1990 |
| GRB2 | 2.13 | 1.89 | 2.11 | 2.043 | 0.133167 | 0.0533 |
| ITGB4 | 2.11 | 2.03 | 2.36 | 2.167 | 0.172143 | 0.0689 |
| DCC | 2.27 | 2.24 | 1.75 | 2.087 | 0.291947 | 0.1168 |
| CCNE1 | 2.03 | 2.19 | 2.44 | 2.220 | 0.20664 | 0.0827 |
| AKT2 | 2.01 | 1.99 | 1.54 | 1.847 | 0.265769 | 0.1063 |
| BBC3 | 2.33 | 1.99 | 1.75 | 2.023 | 0.291433 | 0.1166 |
| BCL2L1 | 2.23 | 1.84 | 2.01 | 2.027 | 0.195533 | 0.0782 |
| NCOA3 | 2.38 | 1.83 | 1.68 | 1.963 | 0.368556 | 0.1474 |
| ZNF217 | 2.18 | 2.12 | 2 | 2.100 | 0.091652 | 0.0367 |
| AURKA | 2.22 | 1.83 | 2.03 | 2.027 | 0.195021 | 0.0780 |
| EEF1A2 | 2.01 | 2.34 | 1.5 | 1.950 | 0.423202 | 0.1693 |
| CRKL | 2.28 | 1.73 | 1.98 | 1.997 | 0.275379 | 0.1102 |

### Example 7

### Breast Milk from the Breast Diagnosed with Cancer is Enriched for Cells with CSC Properties

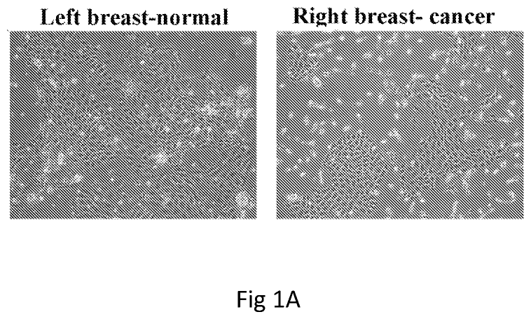

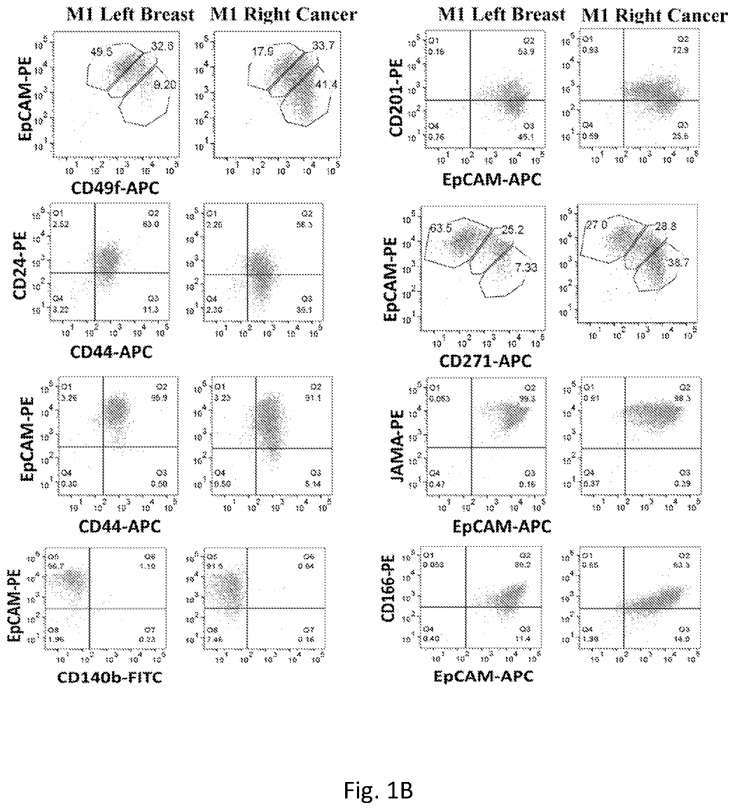

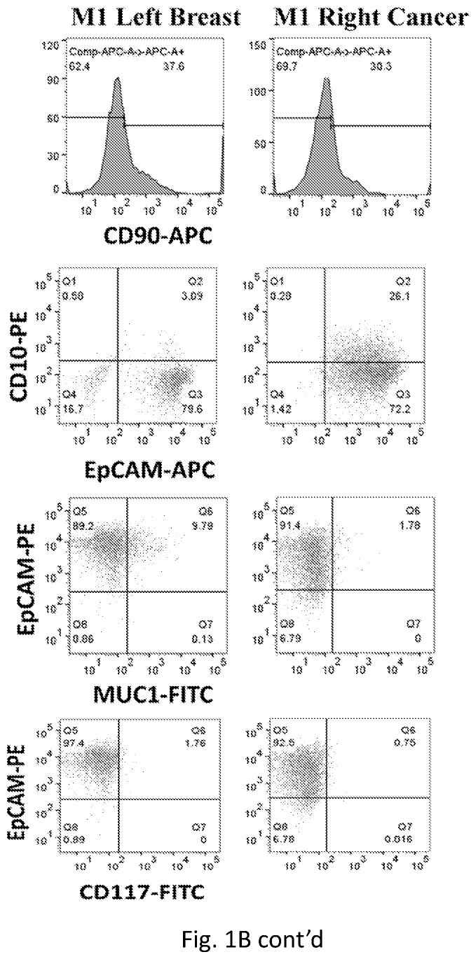

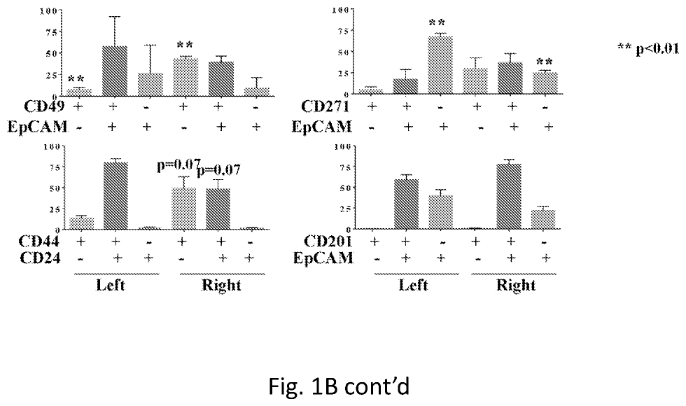

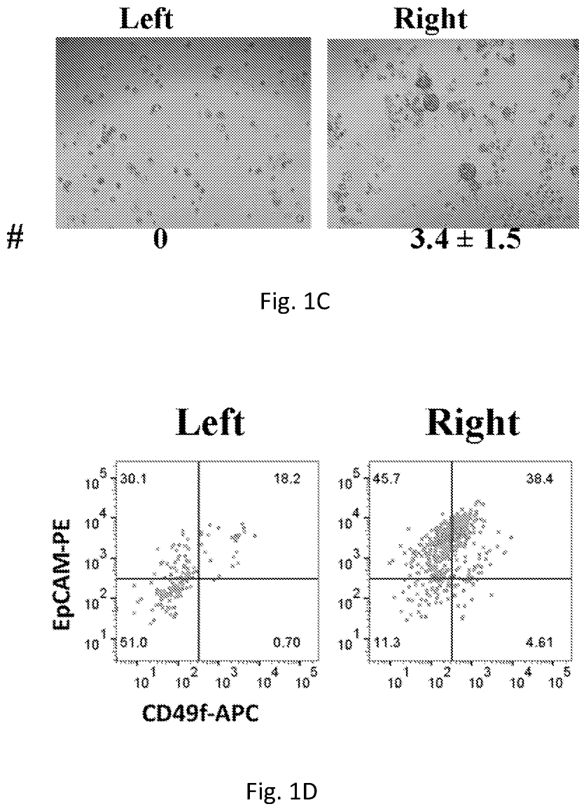

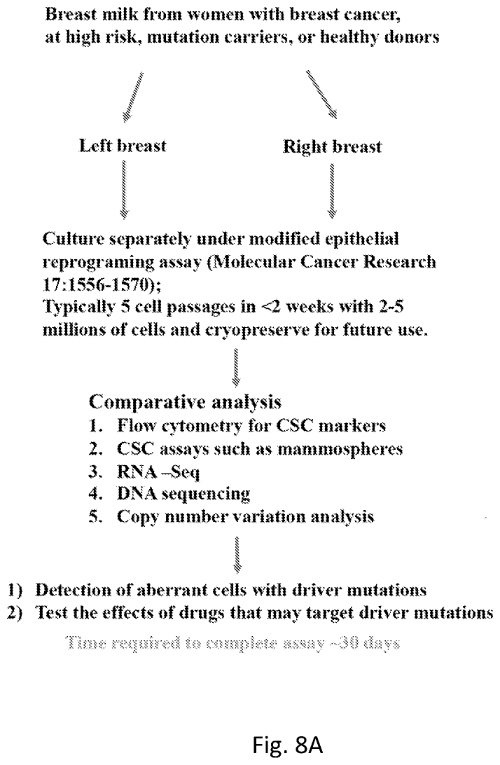

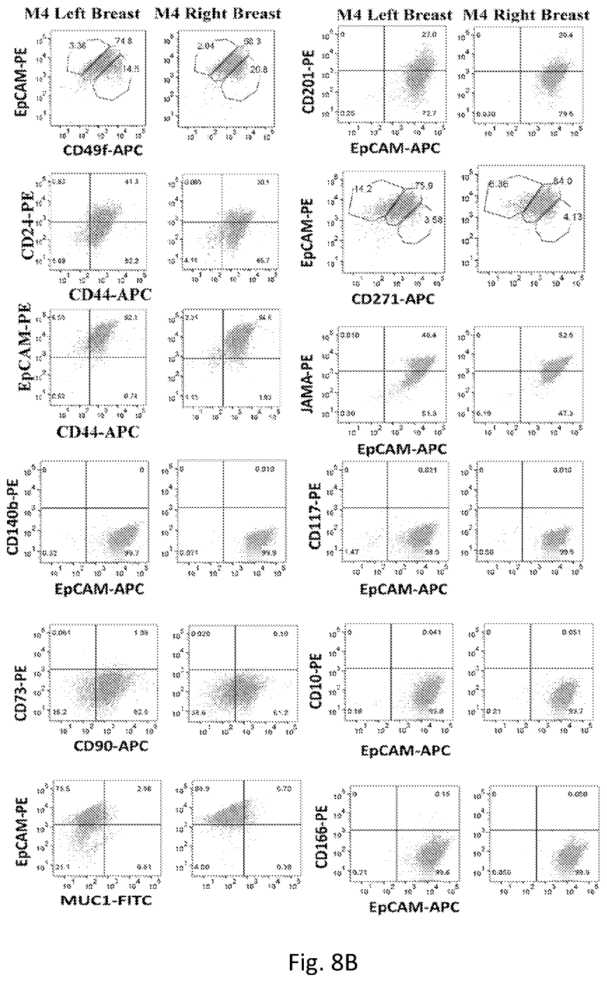

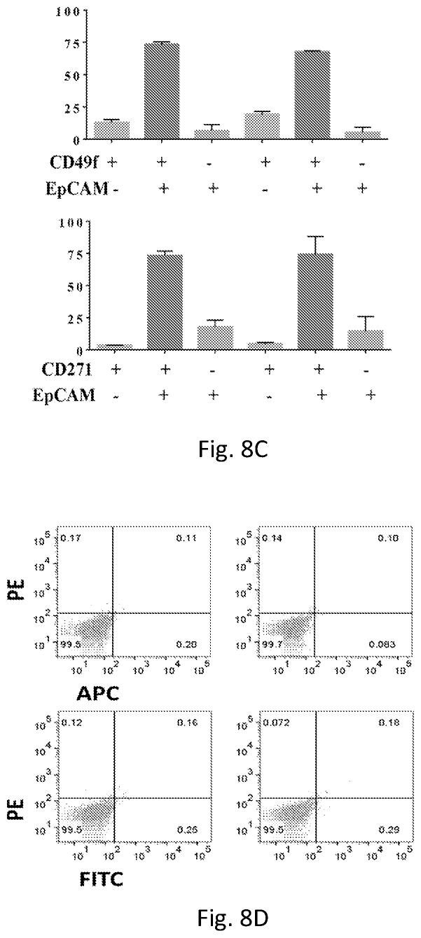

Breast milk was collected from both the breasts prior to initiating any treatment and was cultured using the protocol in Example 1 with the addition of Cipro at 10 μgs/ml to reduce growth of bacteria in milk. Breast epithelial cells grew within a day (FIG. 1A) and cells from both breasts were subjected to flow cytometry using various markers that discriminate stem, luminal progenitor and mature/differentiated cells. A schematic view of the study design is shown in FIG. 8A. CD49f+/EpCAM−, CD49f+/EpCAM+ and CD49f−/EpCAM+ cells are considered to enrich for basal/stem, luminal progenitors, and mature/differentiated cells, respectively. The CD49f/EpCAM staining pattern revealed the presence of higher levels of CD49f+/EpCAM− basal/stem cells in milk of the breast with cancer (right breast) compared to the milk of the breast without cancer (left breast) (FIG. 1B). Cancer stem cells (CSCs), which are defined based on CD44+/CD24− characteristics and whose presence in breast tumors is associated with unfavorable outcomes, were also enriched among cells propagated from milk of the right breast with cancer, although statistical significance was not reached (p=0.07). In general, there was a trend of elevated CSC/progenitor-like features (CD201+/EpCAM+, and CD271+/EpCAM±) in cells derived from milk of the breast with cancer compared to milk of the non-cancerous breast. For example, CD271−/EpCAM+ differentiated cells were lower in the right breast milk-derived cells compared to the left milk-derived cells (p=0.0085). Cells grown from milk from the cancerous breast contained a distinct population of CD10+/EpCAM+ cells. While CD10+/EpCAM− and CD10−/EpCAM+ cells are characterized as basal/myoepithelial and luminal cells, respectively, characteristics of CD10+/EpCAM+ cells are unknown. There was limited fibroblast growth under our culturing condition as cells were negative for CD140b. Additionally, we noted that while cryopreserved cells from the right breast milk could be grown for more than 15 passages, cells from the left breast milk did not survive for long for additional characterization.

### Example 8

### Culturing Breast Milk-Derived Cells

We subjected cells from milk from both breasts to a mammosphere assay to determine whether cells derived from milk from the right breast with cancer show unique properties compared to cells derived from milk from the left breast without cancer. While cells from the left breast milk generated only very small mammospheres (<30 micrometers), larger mammospheres (>50 micrometers) were detected in the right breast milk derived cells (FIG. 1C). Composition of mammospheres was also different as CD49f+/EpCAM+ luminal progenitor cells were enriched in mammospheres of the right breast milk-derived cells compared to mammospheres from cells derived from the left breast milk (FIG. 1D).

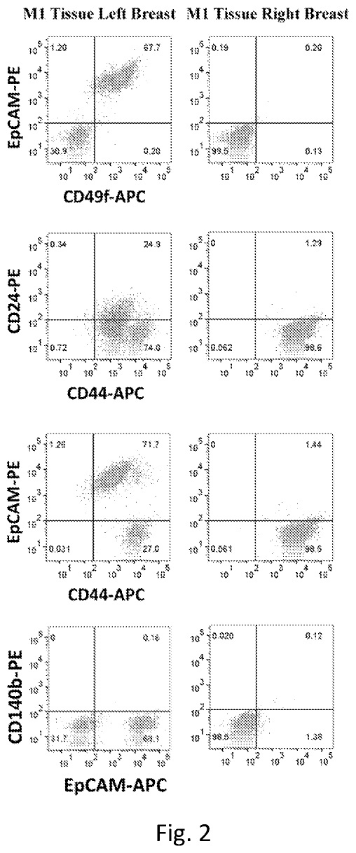

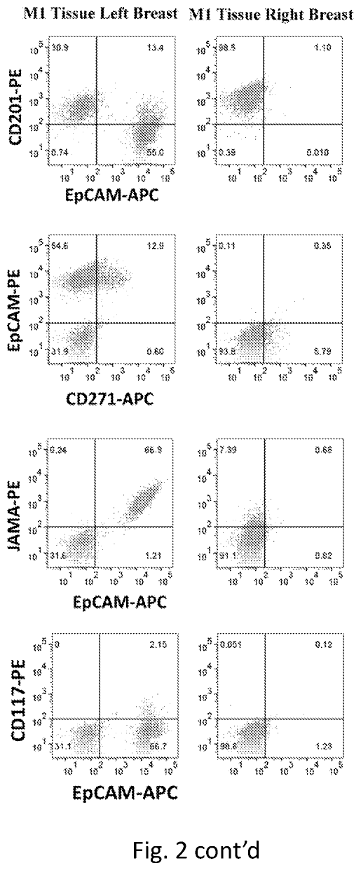

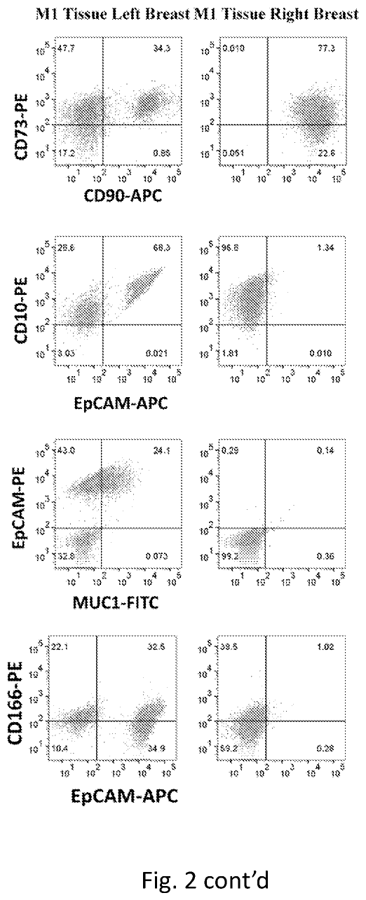

The tumor in the right breast of this donor was a 2.1 cm, moderately differentiated, ER+/PR+(80% nuclear positivity for both), HER2−, grade 2/3, 2/6 lymph node positive, E-cadherin-positive pT2, pN1a invasive ductal carcinoma. E-cadherin positivity indicated that it is not a lobular carcinoma. Treatment consisted of a lumpectomy, followed by chemotherapy, a mastectomy, and radiation. We propagated cells from mastectomy breast tissues. At the time of surgery, there was no evidence of cancer. Flow cytometry showed distinct patterns between the right and left breasts. Cells from the breast with cancer (right) were negative for EpCAM+ epithelial cells but were likely enriched for mesenchymal stem cells as all cells were CD44+ and >75% of cells were CD90+/CD73+. The remaining cells were CD90+/CD73− (FIG. 2). These cells could also be myoepithelial/basal cells as 100% of the cells were CD10+/EpCAM−. At the beginning of culturing, there were small patch of epithelial cells, which were rapidly overtaken by mesenchymal stem-like cells. Cells from the left breast milk and tissue-derived cells from the same breast showed some similarities with respect to CD49f/EpCAM, CD271/EpCAM, Jam-A/EpCAM and MUC1/EpCAM staining patterns but displayed uniqueness with respect to CD44/CD24, CD201/EpCAM and CD10/EpCAM staining patterns. These results suggest that not all cell types of the breast are released into breast milk.

### Example 9

### In a Select Few Cases, Breast Milk from Women with No Known Cancer have Abnormal Cells

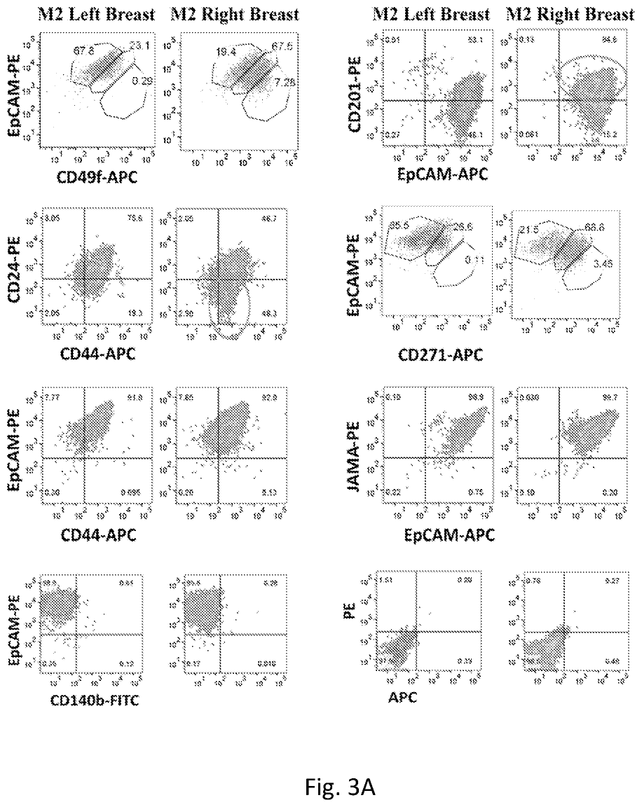

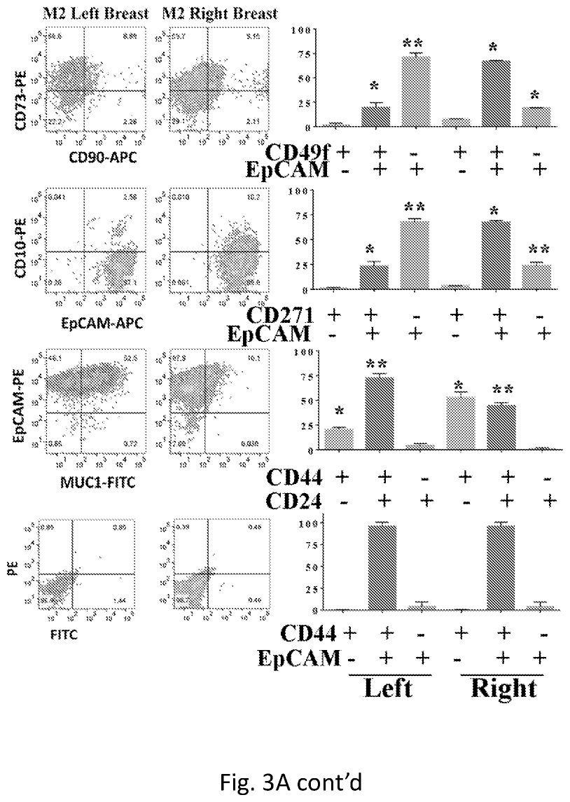

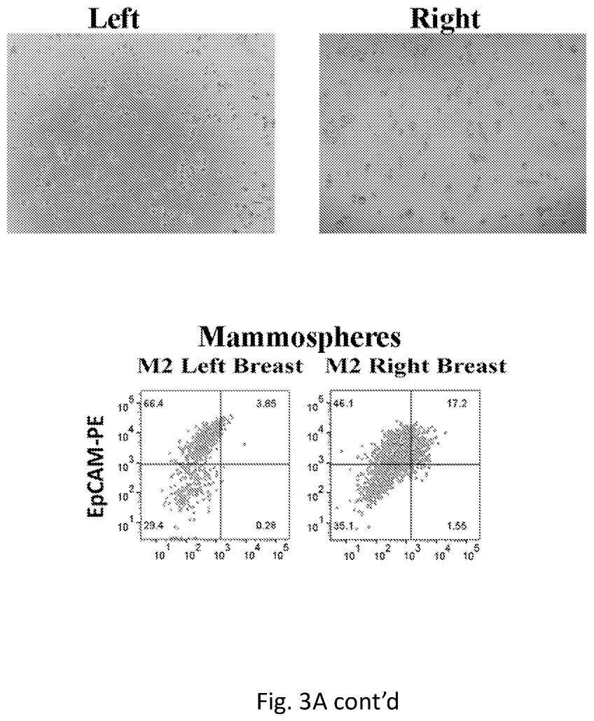

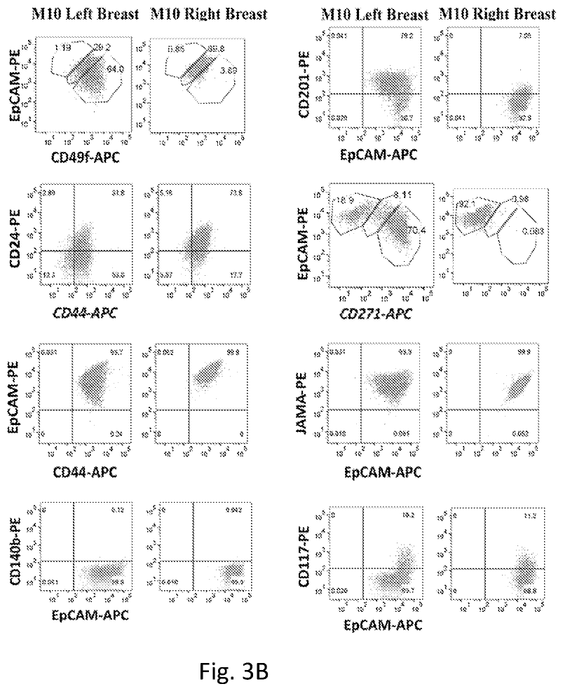

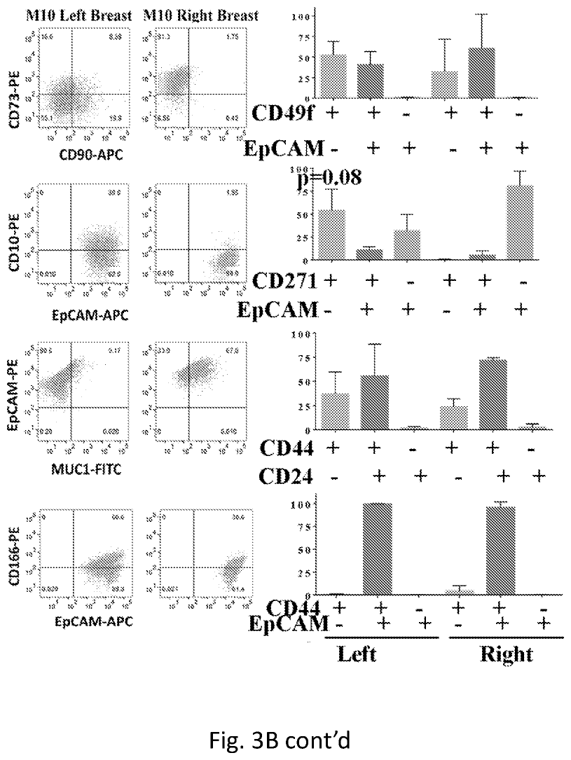

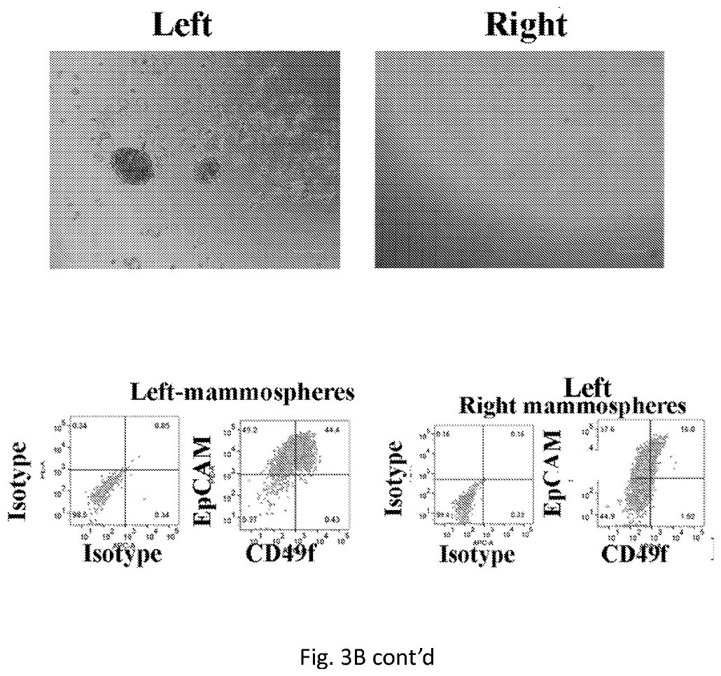

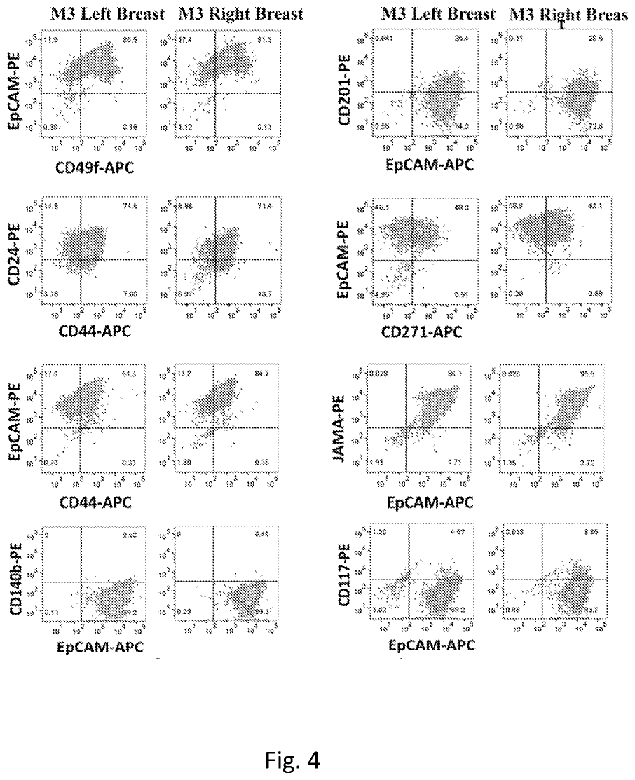

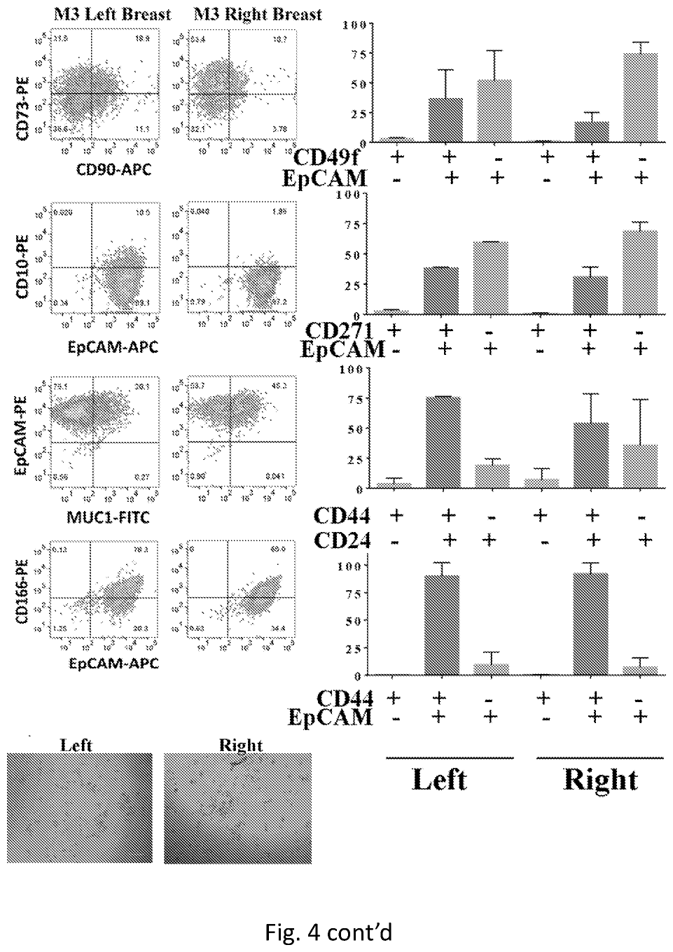

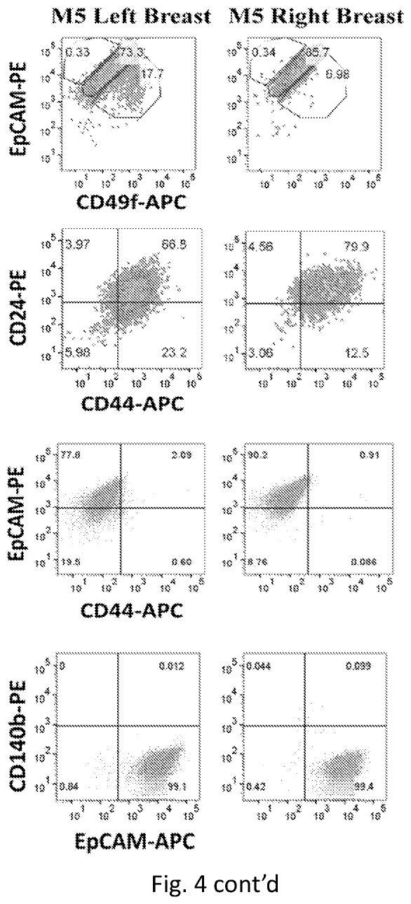

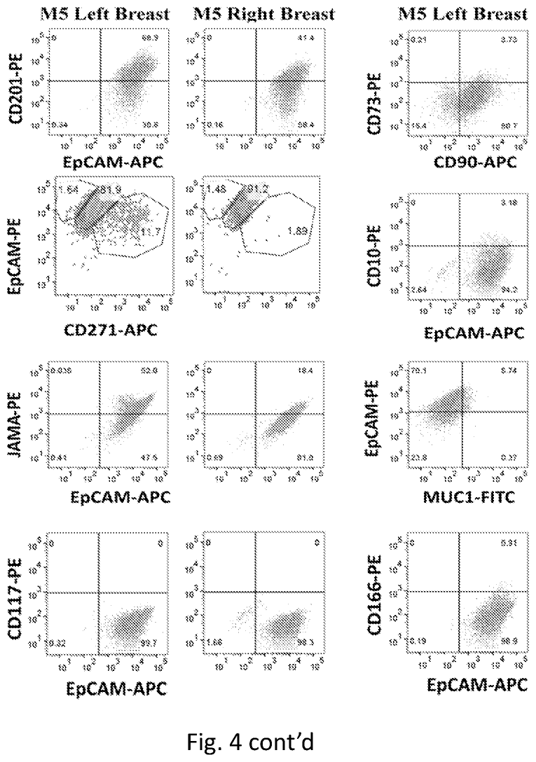

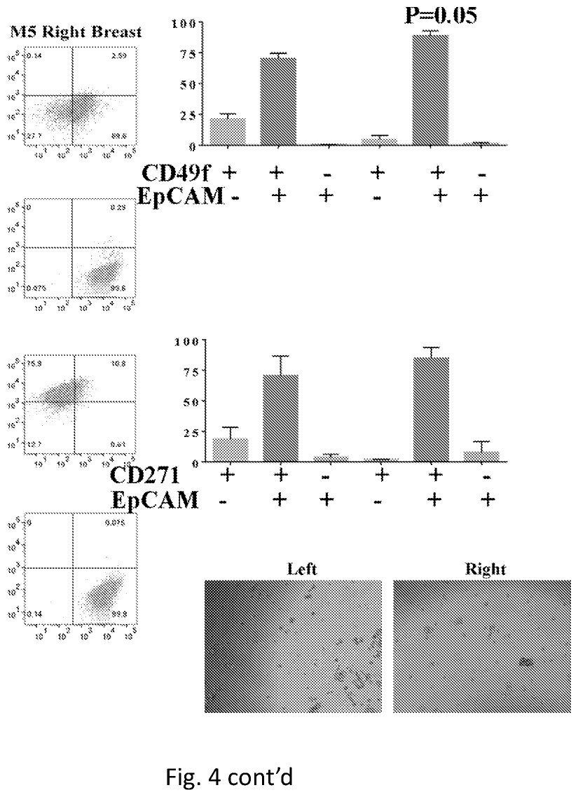

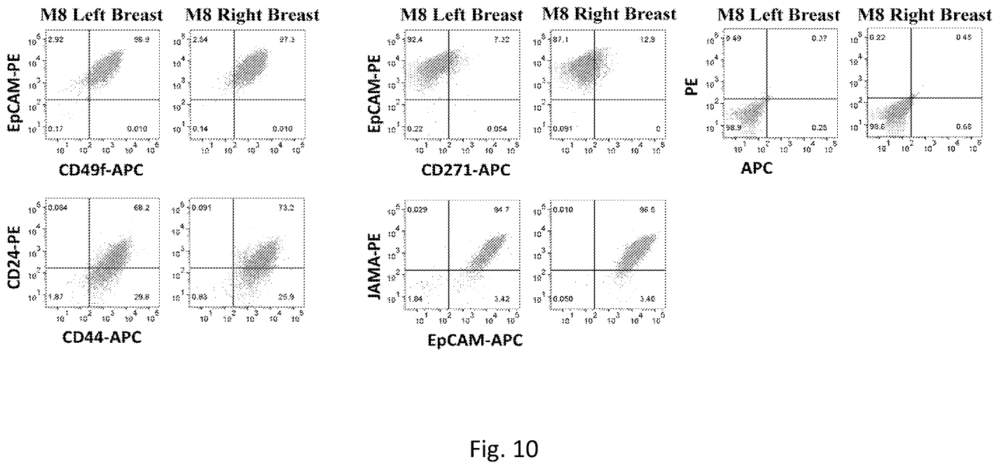

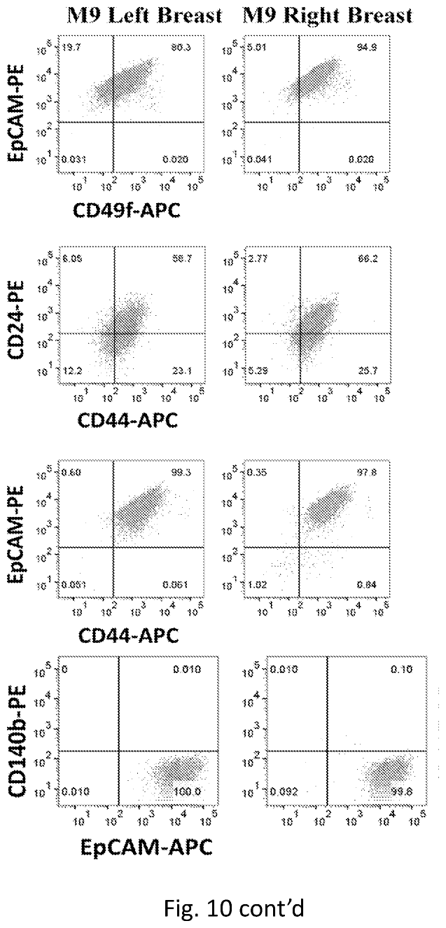

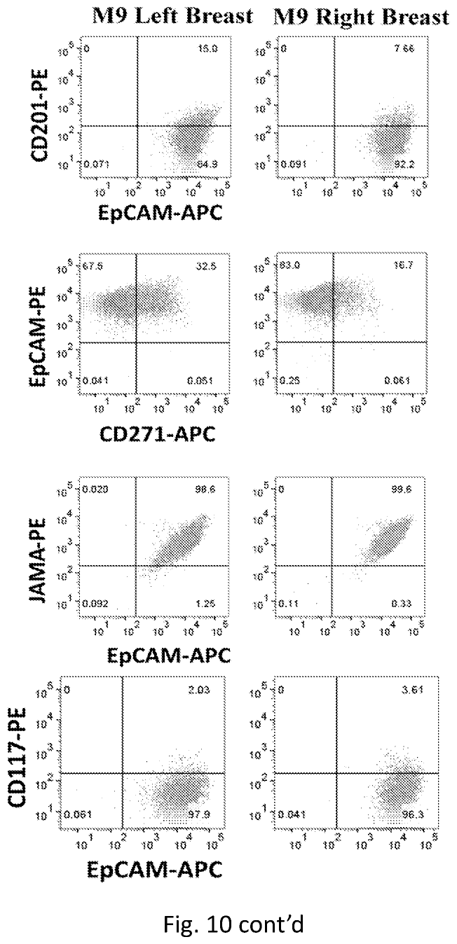

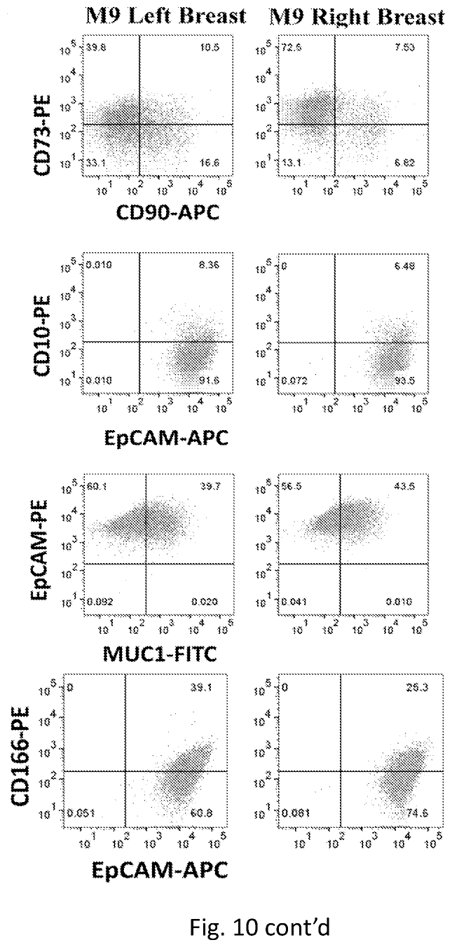

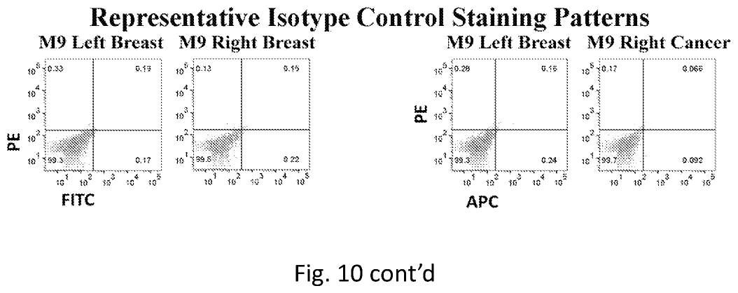

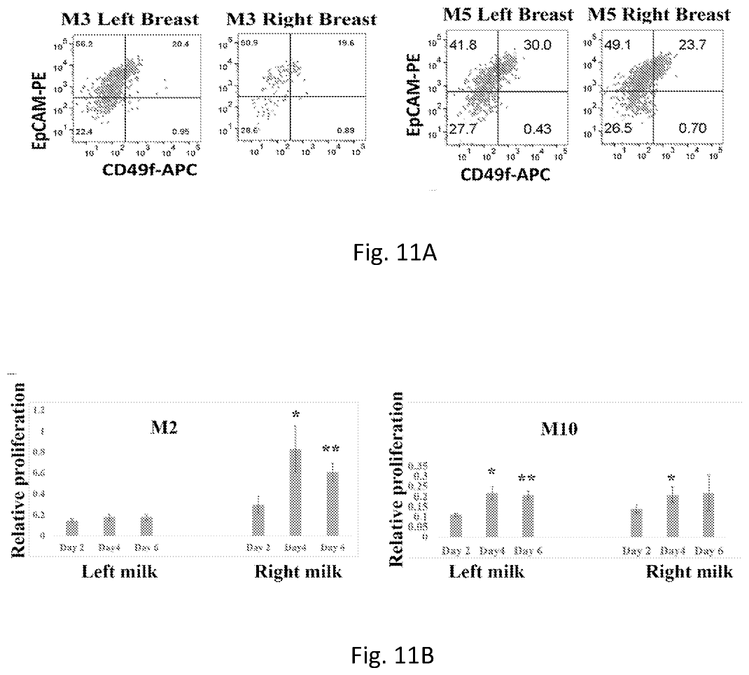

We obtained breast milk from 10 nursing women, propagated cells and characterized cells using flow cytometry. Note that cells from both breasts were cultured for a similar duration and the analyses were done within five passages. Profiles of milk-derived cells from these women are presented in FIG. 3, FIG. 4 and FIG. 8A-8D to FIG. 10. FIGS. 8A-8D and 10 also show representative isotype controls. Since right breast milk-derived cells from donor 1 showed differences in CD49f/EpCAM, CD44/CD24, CD201/EpCAM, CD271/EpCAM and CD10/EpCAM staining patterns compared to left breast milk-derived cells, we paid particular attention to the presence of these differences among breast milk-derived cells from these 10 donors. Breast milk-derived cells from two donors (M2 and M10) showed differences between the right and the left breast with cells from one of the breasts showing similarity to donor 1 right breast milk-derived cells with cancer. For example, cells derived from the right breast milk of M2 compared to cells from the left breast milk contained significantly higher numbers of CD49f+/EpCAM+ luminal progenitors (68% versus 23%, p=0.0061), CD44+/CD24− (52% versus 21%, p=0.02), and CD271+/EpCAM+(69% versus 27%), subpopulations (FIG. 3). Although mammospheres generated from cells of both breasts milk did not show any differences in size, mammospheres generated by cells from the right breast contained higher levels of luminal progenitor cells. Moreover, cells from the right breast milk were highly proliferative compared to the left breast milk-derived cells (FIGS. 11A-11B). In fact, cells from the right breast grew past 15 passages like an established cell line, while from the left breast ceased to grow by 10 passages.

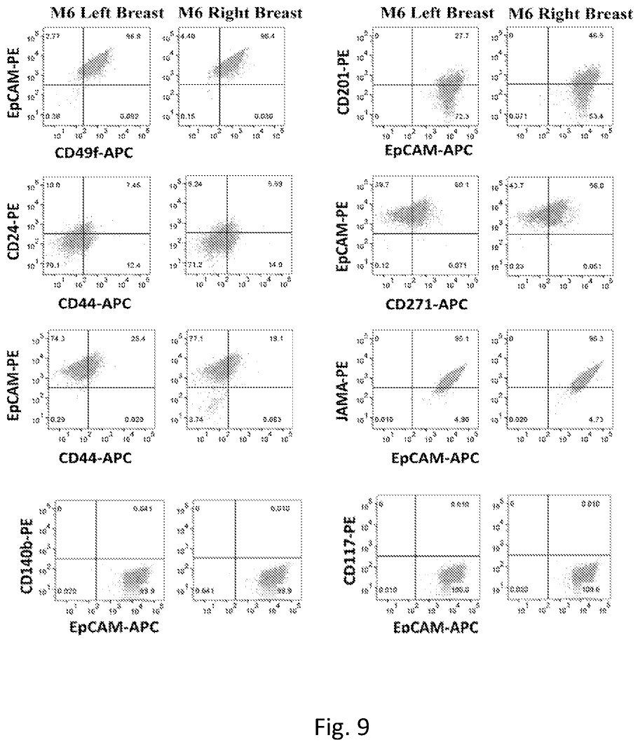

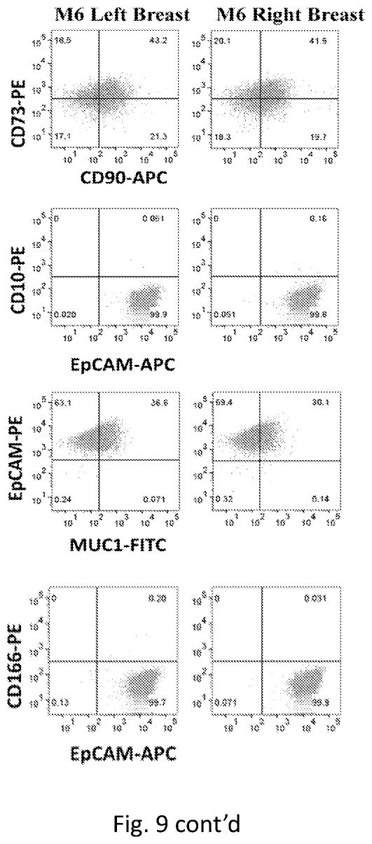

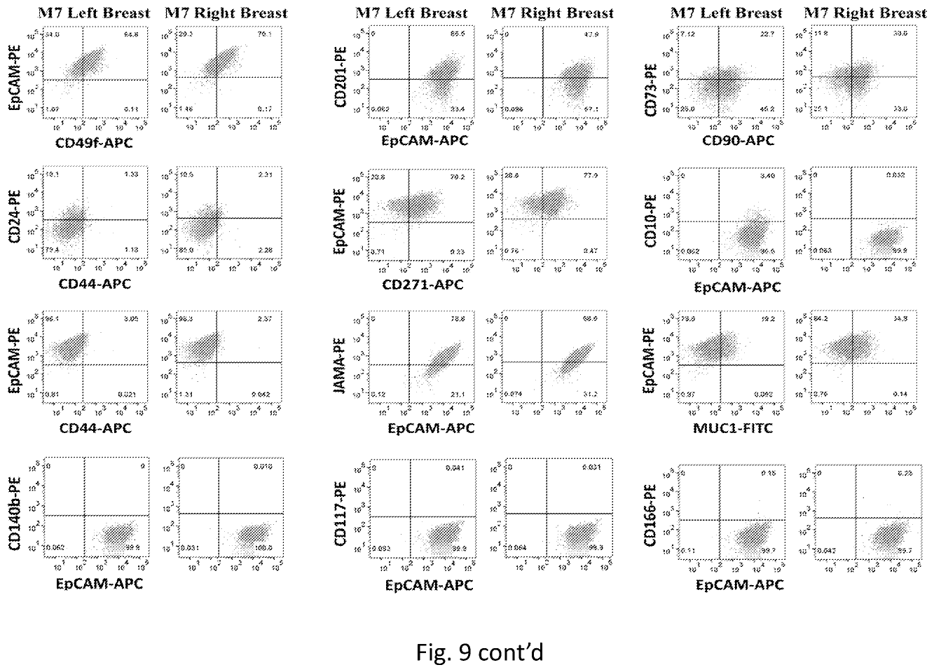

Cells from the left breast milk and the right breast milk of M10 showed differences in subpopulation of CD49f+/EpCAM−, CD44+/CD24− and CD271+/EpCAM− cells, although none of the differences reached statistical difference (FIG. 3). However, similar to cells derived from the right breast of donor 1, cells from the left breast milk in this donor generated larger mammospheres and these mammospheres were enriched for CD49f+/EpCAM+ luminal progenitor cells compared to cells from the right breast. Thus, a phenotypically different population of cells can be obtained from milk of two breasts of the same woman, which may provide the first indication of abnormalities in one of the breasts. We also noted inter-individual differences in cell subpopulations in breast milk-derived cells, similar to differences we reported with cells propagated from the breast tissue of healthy women. For example, while CD271+/EpCAM+ and CD201+/EpCAM+ subpopulations of cells were present at a higher level in milk-derived cells of M5 and M7 (FIG. 9), these subpopulations were lower in M9 (FIG. 10).

### Example 10

### Luminal Characteristics and Differential p63 Expression in Breast Milk-Derived Cells

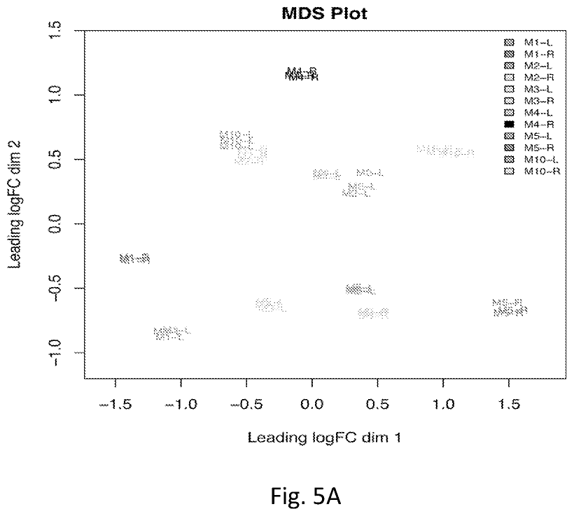

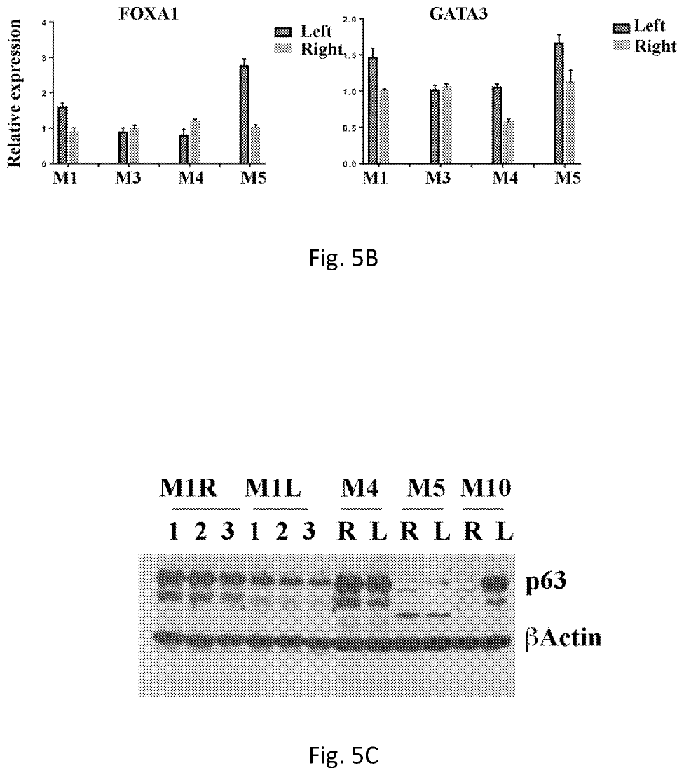

We employed RNA sequencing to determine whether phenotypic changes between breast milk-derived cells of the two breasts of the same individual could be aligned with transcriptomic changes to obtain evidence for the presence of abnormal cells in one of the breasts. RNA sequencing was uninformative as breast milk-derived cells from the right and the left breasts with and without cancer or aberrant cells showed significant differences in gene expression patterns. MDS plots of all samples are shown in FIG. 5A. Nonetheless, RNA sequencing reconfirmed abundant expression of luminal-differentiated cell enriched keratins such as KRT8, KRT18 and KRT19 in milk-derived cells, which suggests that the method of propagating cells enriches for luminal cells including luminal cancer cells and is not biased towards basal/stem cells. Furthermore, breast milk-derived cells expressed abundant levels of luminal markers FOXA1 and GATA3 (FIG. 5B, Ct values of <25 in qRT-PCR). These cells also expressed low levels of ER (Ct value range 29-34), further confirming that our culturing method favors the growth of luminal cells.

We previously demonstrated a close relationship between cancer stem cell phenotype and epithelial to mesenchymal transition (EMT). We examined RNA-seq data for differences in the expression of EMT-associated genes between the right and the left breast milk-derived cells. Expression levels of SNAIL ZEB1, ZEB2, and SOX2 were extremely low, which is consistent with luminal characteristics of cells. SNAI2 (Slug) showed two-fold expression differences between the right breast milk and the left breast milk-derived cells in few cases. The major difference was noted with TP63, which controls self-renewal of breast cancer stem cells through sonic hedgehog pathway. While two-fold differences in expression between the right and the left breast milk-derived cells were common across all samples, the difference was 36-fold in case of M10. We verified these differences at protein levels. Consistent with mRNA data, M1 right breast milk-derived cells contained 2-fold higher levels of p63 protein (FIG. 5C). In case of M10, cells from the left breast milk, which are phenotypically enriched for cancer stem cells, expressed substantially elevated levels of p63 protein compared to cells from the right breast milk. Thus, phenotypic changes in the left breast milk-derived cells in this donor correlates with increased expression of p63.

### Example 11

### Detection of Driver Mutations in Breast Milk-Derived Cells of M1, M2 and M10

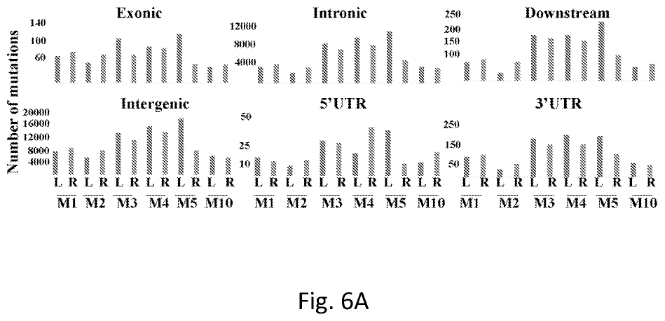

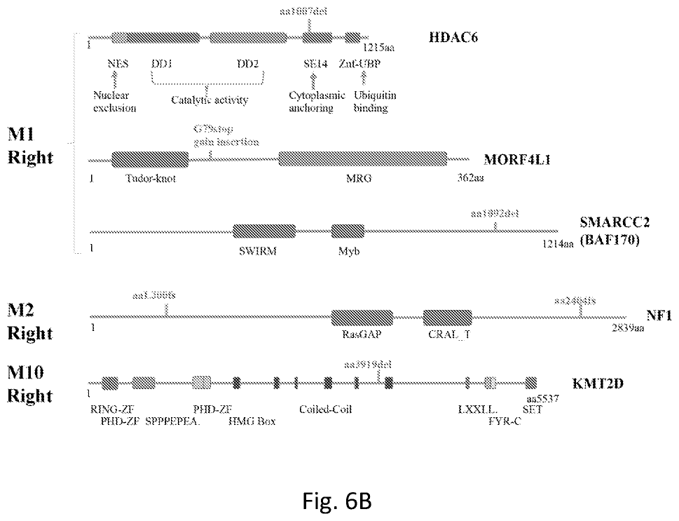

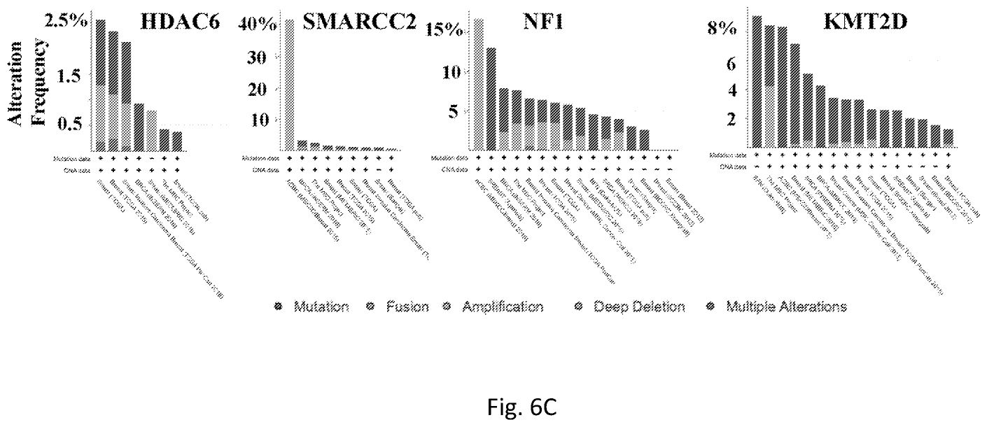

We performed whole genome sequencing of breast milk-derived cell DNA and compared the DNA sequencing results with sequencing data of the corresponding germ line DNA from blood. Summary of the results are presented FIG. 6A. Consistent with a recent report of high level somatic mutations in benign breast tissues with and without proliferative changes, significant number of mutations in exonic, intronic, downstream, untranslated regions as well as intergenic regions were noted in all samples, irrespective of whether these samples are enriched for phenotypically cancer stem-like cells (FIG. 6A). However, a closer look at the exonic mutations revealed the presence of mutations that are considered driver mutations in M1-right, M2-right, and M10-left breast milk-derived cells. Schematic view of these mutations is shown in FIG. 6B. For example, M1-right breast milk-derived cells contained deletion mutation in the SE14 domain of HDAC6. SE14 domain of HDAC6 is required for cytoplasmic retention of this protein. These cells also contained insertion leading to premature termination of MORF4L1, an epigenetic regulator and a member of the BRCA multiprotein complex involved in DNA repair and tumor suppressor activity. A deletion mutation was also observed in SMARCC2 (BAF170), a component of SWI/SNF complex that is defective in 20% of human tumors. Cells from the right breast milk of M2 contained frameshift mutations in NF1 gene, a tumor suppressor that controls ERα activity and mutated in breast cancer. Cells from the left breast milk of M10 contained an amino acid deletion in KMT2D gene, which is also frequently mutated in breast cancer and is a regulator of ERα activity. Analysis of cBioportal database further showed significant alterations of these genes in breast cancer (FIG. 6C).

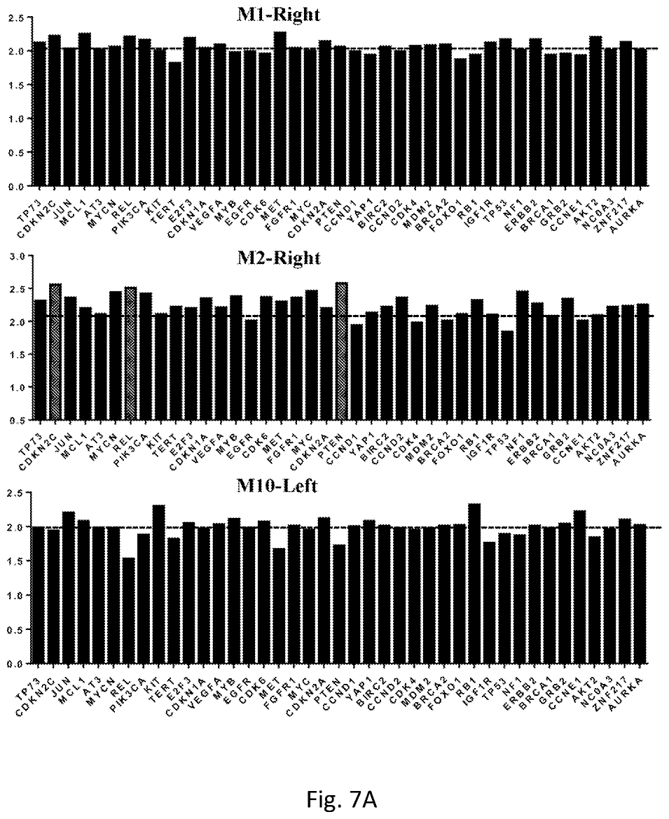

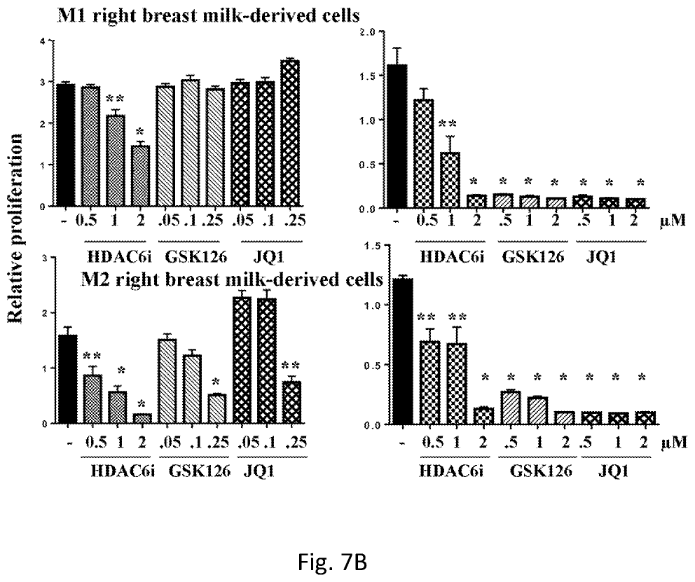

We performed CNV analysis of 87 cancer-relevant genes using DNA from breast milk-derived cells of donors M1. M2 and M10. Breast milk-derived cell DNA from the breast without aberrant cells from the same donor was used as control for comparison. While the right breast milk-derived cells of M1 and the left breast milk-derived cells of M10 did not carry any CNVs in these 87 genes, the right breast milk-derived cells of M2 showed amplification in CDKN2C, PTEN, and REL (FIG. 7A and Tables 1-3). cBioportal database analysis showed amplification of these genes in 1-3% of breast cancers. Collectively, data presented here demonstrate feasibility of using breast milk-derived cells to identify driver mutations, which can be correlated with phenotypic changes in cells.

### Example 12

### Sensitivity of Milk-Derived Cells with Driver Mutations to Targeted Therapies

Prior studies have demonstrated synthetic lethality of SWI/SNF mutated cancers to inhibitors of EZH2 and bromodomains. Similarly, NF1 mutated tumors are susceptible to bromodomain inhibitors. Since cells from the right breast milk of M1 contained mutations in SWI/SNF complex and the right breast milk of M2 had a mutation in NF1 gene, we examined the sensitivity of these cells to EZH2 inhibitor GSK126 and bromodomain inhibitor JQ1. Since cells from right breast milk of M1 contained a mutation in HDAC6 gene, we also tested the effects of the HDAC6 inhibitor CAY10603. Unfortunately, we were unable to use cells from the other breast of the same donors as controls as these cells ceased to proliferate by the time we reached this stage of studies. Cells from the donor M10 also could not be tested because antibiotics used in the culture media failed to suppress milk-resident bacterial growth in this sample. While cells from both donors showed modest sensitivity to the HDAC6 inhibitor, they were extremely sensitive to GSK126 and JQ1 (FIG. 7). Thus, the assays presented in this study not only allow detection of driver mutations in breast milk-derived cells but also demonstrate the feasibility of using these cells for in vitro screening of potential targeted therapies.

### Example 13

This study, initiated due to foresight of a patient, has established a mechanism to identify and characterize aberrant cells in the breast potentially before a radiologic and/or routine breast exam can detect suspicious abnormalities. Cells from breast milk of a donor with known cancer were enriched with CSCs and contained mutations in genes that are linked to breast cancer initiation and/or progression. In 82% of studies that correlated CSC biomarker expression in tumors with outcomes showed an association of CSC marker enrichment with poor overall survival. Since there are no universal CSC biomarkers and characterized biomarkers are associated with different aspects of cancer progression such as drug resistance and metastasis, the method described here would allow for the comprehensive characterization of CSC marker enrichment during early stage of the disease and may potentially aid in monitoring disease progression and making treatment decisions. These phenotypic characterizations coupled with DNA sequencing provided details of earliest lesions associated with breast cancer. For example, in addition to a mutation in HDAC6, which itself is an ERα-regulated gene and mediates estradiol-induced cell migration, DNA from breast milk with ER+/PR+ cancer cells showed mutations in SMARCC2 (BAF170). BAF170 is part of the SWI/SNF complex and the activity of ARID1A-SWI/SNF is essential for luminal cell identity and the control of ER activity and anti-estrogen response in breast cancer. Thus, two major players in ERα functions are aberrant in this cancer.

One in 3000 women are diagnosed with breast cancer during pregnancy or post-partum. These cancers tend to be highly metastatic because remodeling of organs such as liver during lactation provides fertile niche for metastatic cells. In certain instances, as in the case of donor 1, it is possible to propagate cancer cells from breast milk for genomic sequencing and focused screening of drugs that may target driver mutations. Thus, the method described here is beneficial for characterizing at least pregnancy and post-partum breast cancers.

1. A method of detecting a cancer marker in a breast milk sample comprising:
i. extracting nucleotides from the breast milk sample;
ii. analyzing the nucleotides in the breast milk sample for gene expression and breast cancer enriched gene aberrations, and
iii. quantifying the gene expression level and breast cancer enriched gene aberrations of a cancer marker.

2. The method of claim 1, wherein the method further includes a step of amplifying the nucleotides before quantifying.

3. The method of claim 1, wherein the method further comprises detecting the cancer marker based on quantification of the gene expression and breast cancer enriched gene aberrations.

4. The method of claim 3, wherein the cancer marker is p63.

5. The method of claim 4, wherein the gene expression level of p63 is compared to a control sample.

6. The method of claim 5, wherein if the p63 expression level increase is at least 1.5 times higher compared to the control sample, cancer is detected the breast milk sample.

7. The method of claim 5, wherein the control sample is provided from the same patient.

8. A method of culturing breast milk-derived cells comprising:
i. mixing a sample of milk with media; and
ii. plating the milk media mixed sample on a plate;
iii. growing the cells to confluence.

9. The method of claim 8, wherein the media comprises 804G conditioned media.

10. The method of claim 6, wherein the milk to media ratio is 1 to 1.

11. The method of claim 5, wherein the control sample is provided by an average population data set.

12. The method of claim 3, wherein detecting the cancer marker comprises quantifying a copy number of a gene in the nucleotides.

13. The method of claim 12, wherein the copy number of the gene is quantified per ml of sample.

14. The method of claim 3, wherein detecting the cancer marker comprises detecting a mutation in the nucleotides.

15. A method of treating a patient for cancer comprising:
i. mixing a breast milk sample with media to form a sample mixture;
ii. plating the sample mixture on a plate;
iii. allowing cells in the sample mixture to grow to confluence;
iv. extracting nucleotides from the sample mixture;
v. analyzing the nucleotides in the sample mixture for gene expression and breast cancer enriched gene aberrations,
vi. quantifying the gene expression level and breast cancer enriched gene aberrations of a cancer marker,
vi. comparing the quantified expression level and breast cancer enriched gene aberrations of a cancer marker to a control sample, and
vii. treating the patient for cancer if the quantified expression level and breast cancer enriched gene aberrations of the cancer marker is higher than the control sample.

16. The method of claim 15, wherein the cancer marker is p63.

17. The method of claim 16, wherein the method comprises treating the patient for cancer if the p63 expression level increase is at least 1.5 times higher compared to the control sample.

18. The method of claim 15, wherein the control sample is provided from the same patient.

19. The method of claim 15, wherein the control sample is provided by an average population data set.

20. The method of claim 15, wherein the media comprises 804G conditioned media.
